(19) United States
(12) Patent Application Publication
(10) Pub. No.: US 2022/0051794 A1
(43) Pub. Date: Feb. 17, 2022

(54) Methods and Systems for Treating Acne and Other Inflammatory Skin Conditions

(71) Applicant: Alan M DATTNER, Sarasota, FL (US)

(72) Inventor: Alan M DATTNER, Sarasota, FL (US)

(21) Appl. No.: 17/267,019

(22) PCT Filed: Aug. 9, 2019
(86) PCT No.: PCT/US19/46069

§ 371 Date: Feb. 9, 2021

(51) Int. Cl. G16H 50/20; G16H 20/10; G16H 20/60

(52) U.S. Cl. CPC G16H 50/20; G16H 20/60; G16H 20/10

(57) ABSTRACT

Integrative, holistic, whole-system methods and systems for treatment of acne and other inflammatory skin conditions in which multiple patient data points are collected and/or organized and/or interpreted, and relationships between multiple patient data points are determined, and the relationships are applied by formulating and adjusting treatment options based on the relationships, and structuring a treatment protocol based on treatment options, and administering the treatment protocol, and monitoring patient for response to the treatment protocol, wherein these steps are done in view of underlying causal factors of acne and other inflammatory skin conditions, and wherein one or more of the steps are iterated.

### CROSS-REFERENCE TO RELATED APPLICATION

This application is a national stage application, filed under 35 U.S.C. Section 371(c), of International Patent Application No. PCT/US2019/046069, with an International filing date of Aug. 9, 2019, which is incorporated by reference herein in its entirety, which includes the substitute sheets filed on Nov. 16, 2019 as fifty-two pages of specification, claims and abstract and three pages of drawings.

### BACKGROUND

Acne and other inflammatory skin conditions afflict millions of people worldwide and result in physical discomfort and pain as well as diminished emotional well-being and result in disfigurement and physical discomfort. Methods for treating acne and other inflammatory skin conditions that are known in the art include using topical compositions such as those containing retinoids, antibiotics combined with benzoyl peroxide, oral antibiotics such as tetracycline and salicylic acid, combined oral contraceptives such as combined estrogen and progestin, orally-administered isotretinoin, light therapy such as laser light, chemical peels, extraction of whiteheads or blackheads, injected steroid drugs, ultrasound energy, cleansing devices such as oscillating brushes, and biologic agents such as tumor necrosis factors alpha inhibitors.

Known methods for treating acne and other inflammatory skin conditions may be subject to multiple disadvantages and problems, including causing unwanted side-effects, failing to address underlying causal factors of acne and/or inflammatory skin conditions, being ineffective, addressing only symptoms, negatively altering the intestinal and/or skin microbiome, favoring yeast overgrowth in the intestines, failing to address ileocecal valve dysfunction and/or leaky gut issues, approaching treatment from a limited and/or non-integrated and/or piecemeal perspective, overuse of antibiotics, overuse of birth control pills, making unwanted epigenetic changes, as well as scarring, redness, dryness, discoloration, tenderness, and irritation of the skin, skin sensitivity to the sun, weight gain, nausea, abdominal pain, bloating, increased risk of blood clots, ulcerative colitis, increased risk of depression and suicide, and severe birth defects. Thus, there exists a need for novel methods and systems for treating acne and other inflammatory skin conditions that are not subject to multiple disadvantages and problems.

### SUMMARY OF INVENTION

An object of the invention is to provide methods and systems for treating acne and other inflammatory skin conditions, as readily recognized and understood by one of ordinary skill in the art based upon this disclosure, inter alia, by addressing underlying causal factors of acne and inflammatory skin conditions, by treating the patient safely, effectively and without unwanted side-effects, by providing methods and systems for treating acne and other inflammatory skin conditions that treat in an integrative, holistic, whole-system way, and as described in the multiple aspects herein, which novel methods and systems of the invention solved the multiple disadvantages and problems of known methods as described above, including one or more of reducing unwanted side-effects, restoring balance to the intestinal and/or skin microbiome, reducing and/or eliminating yeast overgrowth in the intestines, addressing and/or correcting ileocecal valve dysfunction and/or leaky gut issues, reducing use of antibiotics, reducing use of birth control pills, and reducing weight gain, reducing and/or eliminating nausea and abdominal pain and bloating. Another object of the invention is to provide methods and systems for treating acne and other inflammatory skin conditions, as readily recognized and understood by one of ordinary skill in the art based upon this disclosure, inter alia, by utilizing the steps described herein to provide safe, effective, integrative, holistic, whole-system methods and systems for treating acne and other inflammatory skin conditions, by recognizing and focusing therapeutically on the underlying casual factors as described herein especially, for example but not limited to, in diet, digestive system and environment, by enhancing digestive breakdown of food molecules and/or increasing cleavage of them into fully-digested and immunologically unrecognizable parts, by recognizing and focusing on reducing and/or eliminating Candida and other yeast overgrowth and foods that feed such organisms such as supplying sugars and/or simple carbohydrates for growth and tolerize them; that is, induce immunological tolerance of the gut immune system to allow for such overgrowth, by recognizing and focusing on reducing and/or eliminating food allergens, Candida, and gluten, as described herein, that foster leaky gut, by recognizing and focusing on reducing insulin-like growth factor-1 by, for example but not limited to, reducing dietary sugar and by supplying nutrients such as chromium and supplying herbs such as fenugreek to improve insulin sensitivity and reduce insulin levels, by recognizing and utilizing tongue and/or frenulum diagnosis and/or Applied Kinesiology that is Professional Applied Kinesiology to obtain multiple patient data points regarding organ function, such as for example without limitation by testing the quadriceps for weakness along with the neuro-lymphatic points under the lower rib cage and if stronger then by recognizing and understanding the use of digestive enzymes indicated, by recognizing and focusing on enhancing patient digestion with digestive enzymes, by recognizing and focusing on enhancing liver function using silymarin and other related liver-enhancing herbs, by recognizing and focusing on correcting constipation with using water, fiber, magnesium and/or vitamin C and/or Indian echinacea that is Andrographis and/or usnea such as that from Spanish moss and/or Amla that is Amlaki that is Indian gooseberry that is Emblica officinalis that is Phyllanthus emblica along with aloe as needed, by recognizing and focusing on reducing inflammation of the gut lining by a pro-inflammatory microbiome by soluble fiber, anti-yeast herbs, and/or supplements such as caprylic acid and undecanoic acid, Nystatin that is the polyene macrolide antifungal as needed, and/or a combination of berberine, coptis, and Scutellaria baicelensis which contains Baicalin as an antimicrobial, by testing as needed stool flora, by recognizing and focusing on coating and/or repairing the gut and gut lining with for example the dietary addition of glutamine and/or mucoid substances and/or elimination of NSAIDs, only after Candida is under control that is no longer overgrowing as assessed by diet history and digestive history and strengthening of muscle response when touching over acupuncture points lung one and lung two, by recognizing and focusing on herbs that have anti-inflammatory effects such as for example without limitation a flavone glycoside such as Baicalin, by recognizing in cases of female patients with hormonal-related acne or other inflammatory skin conditions to focus on use of pyridoxal-5-phosphate, calcium-D-glucarate, chaste tree extract that is chasteberry and indole-3-carbinol, and by recognizing in cases where there is accompanying autoimmune conditions such as associated with Epstein-Barr virus that is EBV and/or Cytomegalovirus that is CMV to focus on administering one or more antivirals, as further defined herein, and including for example immune-stimulating mushrooms such as for example reishi mushrooms that are utilized when other anti-virals are not tolerated and/or fail to produce results, such as lauricidin that is monolaurin along with administering an algal concentrate such as for example form red algae and/or an algal concentrate containing one or more of an allophycocyanin-rich extract of microalgae such as spirulina and/or chlorella and/or a C-phycocyanin-containing mixture of microalgae such as spirulina and/or chlorella and/or a growth factor-containing mixture derived from microalgae such as spirulina and/or chlorella, wherein such administration leads to a method wherein for example but not limited to the inhibition of the virus entry into cells such that virus circulating in the bloodstream is increased so that it is more easily addressed using antivirals, as known by a person of ordinary skill in the art and as further defined herein, and by recognizing in cases of patient elevated test results for Epstein-Barr virus and/or Cytomegalovirus titers to focus on administering one or more antivirals, as known by a person of ordinary skill in the art and as further defined herein, such as lauricidin that is monolaurin along with administering an algal concentrate and/or such as for example from red algae and/or an algal concentrate containing one or more of an allophycocyanin-rich extract of microalgae such as spirulina and/or chlorella and/or a C-phycocyanin-containing mixture of microalgae such as spirulina and/or chlorella and/or a growth factor-containing mixture derived from microalgae such as spirulina and/or chlorella, wherein such administration leads to a method wherein for example but not limited to the inhibition of the virus entry into cells such that virus circulating in the bloodstream is increased so that it is more easily addressed using antivirals, as known by a person of ordinary skill in the art and as further defined herein. Yet another object of the invention, as readily recognized and understood by one of ordinary skill in the art based upon this disclosure, is to provide safe, effective, integrative, holistic, whole-system methods and systems for treating acne and other inflammatory skin conditions that includes, inter alia, the steps of collecting and/or organizing and/or interpreting multiple patient data points, for example but not limited to, patient queries and/or patient histories and/or medical testing and/or observation of patient and/or examination of patient and/or evaluation of patient and/or conventional medicine wherein the term “conventional medicine”, as recognized and understood by one of ordinary skill in the art, also includes meaning, as appropriate given the context of the usage, Western medicine or mainstream medicine, and/or traditional medicine and/or complementary medicine and/or alternative medicine and/or causal factors of acne and/or causal factors of inflammatory skin conditions, determining relationships between multiple patient data points, applying relationships between multiple patient data, formulating treatment options, adjusting treatment options, structuring a treatment protocol, administering a treatment protocol, monitoring patient for response to administered treatment protocol, optionally reiterating one or more of these steps of collecting, organizing, interpreting, determining, applying, formulating, adjusting, structuring, administering, which novel methods and systems of the invention solve the multiple disadvantages and problems of known methods as described above, wherein digestive tract sources for PSU and/or sebaceous gland inflammation are addressed to reduce inflammation namely to reducing and/or removing inflammatory triggers from the skin and/or lymphatics and/or the blood supply for the small intestines by, for example but not limited to, the use of herbs that may contain saponins, including for example but not limited to burdock root and seed and others known to a person of ordinary skill in the art, and wherein there is a recognition and focus on hormonal rebalancing by assisting to promote excretion of hormonally-active and toxic substances from the body such as calcium-D-glucarate that helps prevent deconjugation of for example estrogen-glucuronide and/or other glucuronide-bound toxins and/or pseudo-estrogens whereupon they are excreted from the body and thereby ameliorating hormonal-mediated acne and other inflammatory skin conditions without the use of hormones, and wherein by recognizing and focusing first on addressing the underlying causal factors of acne and other inflammatory skin conditions the use of topical treatments can be reduced and/or eliminated, and wherein by recognizing and focusing on the causal factors of acne and other inflammatory skin conditions as a way to reduce and/or eliminate chronic viral infections such as Epstein-Barr virus and/or Cytomegalovirus that dysregulate the immune system and favor inflammation in the patient, by administering to the patient one or more antivirals, as known by a person of ordinary skill in the art, in some embodiments of the invention, for example without limitation, administering to the patient lauricidin that is monolaurin along with administering to the patient an algal concentrate containing one or more of an allophycocyanin-rich extract of microalgae such as spirulina and/or chlorella and/or a C-phycocyanin-containing mixture of microalgae such as spirulina and/or chlorella and/or a growth factor-containing mixture derived from microalgae such as spirulina and/or chlorella, wherein such administration leading to a method wherein for example but limited to the inhibition of the virus entry into cells such that virus circulating in the bloodstream and/or lymphatic system is at least maintained and/or increased so that the virus that is circulating and/or virus that is not made entry into the cells is more easily addressed using antivirals, as known by a person of ordinary skill in the art and as further defined herein in some embodiments of the invention, and wherein such administration both protects patient cells from viral entry and kills circulating viruses in the bloodstream, and by utilizing the steps described herein to provide safe, effective, integrative, holistic, whole-system methods and systems for treating acne and other inflammatory skin conditions wherein the term “whole-system” as recognized and understood by one of ordinary skill in the art based upon the disclosure herein also includes meaning the tremendous paradigm shift as described herein to approaching the treatment of acne and other inflammatory skin conditions, by for example, addressing the causal factors of acne and other inflammatory skin conditions and/or considering the collateral damage, as defined herein, associated with a given treatment protocol, and in one aspect, the invention provides methods and systems for treating acne and other inflammatory skin conditions for patients who fail repeated attempts at all manner of known conventional and/or alternative treatments and are subsequently worse off from the long-term side effects of such treatments, and benefit greatly from the advantages of the methods and systems for treating acne and other inflammatory skin conditions as described and disclosed herein.

In one aspect, the invention provides methods and systems for treating acne and other inflammatory skin conditions utilizing one or more antivirals wherein the term “antiviral”, as recognized and understood by one of ordinary skill in the art, also includes meaning, as appropriate given the context of the usage, certain antivirals that include for example without limitation, cytokine or other inflammatory inhibitors and/or modulators such as statins, melatonin; cathepsin-B inhibitors such as salianolic acid B; cathepsin-L inhibitors such as drynariae rhizoma, chelidocystatin, vitamin D-3; helicase nonstructural protein 13 inhibitors such as radix isatidis from the root of isatis indigotica, silvestrol a natural product of the flavagline family found in bark of the genus Aglaia trees; mannose-binding lectin that is mannose-binding protein gene agonists such as beta glucans, Chinese chive, garlic, root of Japanese angelica acutiloba root, shallots, sarsparilla, vitamin A; anti-viral lectins such as garlic, leek, onion, jackfruit seeds, black elder, isolates from the snowdrop bulb for example those known as Galanthus nivalis lectin, shallots, isolates from smilax rhizome that is sarsparilla, especially those found native in China; bitter melon that is Momordica charantia; antiviral natural products and/or antiviral plant products such as deglycyrrhizinated licorice that is DGL, phellodendron cortex, vitamin C, a species of elderberry known as sambucus formosana Nakai, caffeic acid, reservatrol, acanthopanacis cortex, sanguisorbae radix, selenium, torilidis fructus from fruits of Torilis japonica Decadolle and/or the preparation mainly produced as fructus torilis in the Chinese provinces of Hunan, Guangxi and Fujian, compounds found in Moms alba juice and/or seed such as gallic acid and/or antivirals compounds in and/or derived from for example stinging nettle from the genus Urtica that is Urtica dioica or Urtica galeopsifoli and/or antivirals found in the herbs and/or plants of the genus Astragalus and/or valproic acid and/or butyrate and/or the naturally-occurring anti-inflammatory epoxomicin and/or lectins that is plant agglutinin especially depending on their sugar moiety including for example mannose and N-acetyl glucosamine; and furthermore the antivirals disclosed herein, as recognized and understood by one of ordinary skill in the art, and including the meanings disclosed herein, and as appropriate given the context of the usage, are utilized in some embodiments of the invention wherein an algal concentrate such as for example from red algae and/or an algal-derived product such as for example an antiviral lectin from red algae and/or an algal concentrate containing one or more of an allophycocyanin-rich extract of microalgae such as spirulina and/or chlorella and/or a C-phycocyanin-containing mixture of microalgae such as spirulina and/or chlorella and/or a growth factor-containing mixture derived from microalgae such as spirulina and/or chlorella is administered and wherein such administration using the methods and systems of the invention results in, for example and without limitation, the inhibition of a virus entry into cells such that virus circulating in the bloodstream and/or lymphatic system is increased so that the virus is more easily attacked by administering antivirals including, as one example and without limitation, gallic acid from Moms alba, and in some embodiments of the invention the antiviral is utilized against for example without limitation human herpes viruses, Dengue virus, influenza virus such as H1N1 and H5N1, picornaviruses such as Coxsackievirus A16, enterovirus 71, coronaviruses, and along with a lipid extract, Hepatitis B, wherein the antiviral is an algal concentrate such as for example from red algae and/or an algal concentrate that is an extract from Spirulina and for example especially a multi-hundred concentrate, for example containing one or more of an allophycocyanin-rich extract of microalgae such as spirulina and/or chlorella and/or a C-phycocyanin-containing mixture of microalgae such as spirulina and/or chlorella and/or a growth factor-containing mixture derived from microalgae such as spirulina and/or chlorella, and wherein the antiviral algal concentrate, as described herein, is part of a synergistic combination with lauricidin, also includes meaning, as appropriate given the context of the usage, certain antivirals that are utilized for patients with coconut allergies such as for example Andrographis, berberine, coptis, Isatis, and/or immuno-enhancing antiviral mushrooms such as reishi.

In one aspect, the invention provides methods and systems for treatment of acne and other inflammatory skin conditions that is best described as a paradigm shift in the treatment of acne and other inflammatory skin conditions in a whole system way, that is, for example, a 360-degree approach to treatment including efforts by both the person treating the patient and the patient, and that includes for example input and/or iterative input from an understanding of and/or knowledge of every relevant aspects of the patient contributing to the causal factors of acne and other inflammatory skin conditions and to ferret out of that constellation of causal factors that is collect multiple patient data points and/or input and/or iterative input that is organized and then interpreting multiple patient data points to determine relationships between the multiple patient data points and applying relationships between the multiple patient data points by formulating treatment options based on relationships between the multiple patient data points and optionally adjusting treatment options based on relationships between the multiple patient data points and structuring a treatment protocol based on treatment options and administering treatment protocol to a patient and then monitoring patient response to treatment protocol, all these steps this creating a synergistic physiological shift that improves the acne and/or other inflammatory skin condition and the accompanying pathophysiological issue of which acne and/or other inflammatory skin conditions are a symptom. In one aspect, the invention provides methods and systems for treatment of acne and other inflammatory skin conditions that includes a commitment by the patient to change many aspects of their lives to contribute to the treatment of the acne and/or other inflammatory skin conditions. In one aspect, the invention provides methods and systems for treatment of acne and other inflammatory skin conditions that over time results in a combination of multiple modalities as recognized and understood by one of ordinary skill in the art based upon the disclosure herein, and which resultant synergistic combination reaches an effective critical mass, wherein the term critical mass, as recognized and understood by one of ordinary skill in the art and as described and disclosed herein includes meaning employing over a period of time the methods and systems of the invention whereby is accumulated over time a critical mass of safe, effective, integrative, holistic, whole-system methods and systems for treating acne and inflammatory skin conditions and inflammation of the body, and when sufficient and multiple remedies are implemented whether to the patient environment, microbiome, digestive system, processing and excretory systems, hormonal systems, diet, nutrition, patient metabolism, patient behaviors and/or emotions and/or habits and or any addictive malfunctions, which effective critical mass contributes to restoring the patient homeostasis that reduces and/or eliminates the acne and/or other inflammatory skin conditions, for example without limitation, on the perifollicular apparatus, its flora, tissues, organs, and eliminative sebaceous contents, and which effective critical mass is reached upon collecting multiple patient data points and/or input and/or iterative input and organizing and then interpreting multiple patient data points to determine relationships between the multiple patient data points, all from a vastly broader perspective than found in the prior art methods of treatment, and including observations using, for example without limitation as described herein, physical and energetic techniques, to understand organ function and integrating these observations into a multi-dimensional therapeutic protocol based upon relationships between the multiple patient data points and formulating treatment options based on relationships between the multiple patient data points and optionally adjusting treatment options based on relationships between the multiple patient data points and structuring a treatment protocol based on treatment options and administering treatment protocol to a patient and then monitoring patient response to treatment protocol, wherein the patient benefits from improved health and wellbeing at least for the reason that the underlying causal factors of the inflammation are also addressed.

In one aspect, the invention provides methods and systems for treating acne and other inflammatory skin conditions, which inflammatory skin conditions include Acne vulgaris, Pityriasis rosea, alopecia areata, vitiligo, Pityriasis lichenoides chronica, Hidradenitis suppurativa, atopic dermatitis, contact dermatitis, folliculitis, psoriasis, eczema, seborrhea, and other inflammatory skin conditions as described and disclosed herein, dermatological diseases and disorders, and also including inflammatory conditions that are treated by providing safe, effective, integrative, holistic, whole-system methods and systems of the invention that includes, inter alia, the steps of collecting and/or organizing and/or interpreting multiple patient data points, for example but not limited to, patient queries and/or patient histories and/or medical testing and/or observation of patient and/or examination of patient and/or evaluation of patient and/or conventional medicine and/or traditional medicine and/or complementary medicine and/or alternative medicine and/or causal factors of acne and/or causal factors of inflammatory skin conditions, determining relationships between multiple patient data points, applying relationships between multiple patient data, formulating treatment options, adjusting treatment options, structuring a treatment protocol, administering a treatment protocol, monitoring patient for response to administered treatment protocol, optionally reiterating one or more of these steps of collecting, organizing, interpreting, determining, applying, formulating, adjusting, structuring, administering, wherein such inflammatory conditions, when treated using the novel methods and systems of the invention, are improved at least for the reason that the underlying causal factors of the inflammation are also addressed.

In another aspect, the invention disclosed herein in the specification, examples, drawings, and claims, produced unexpected and positive results in contrast to and thereby solving one or more of the multiple disadvantages and problems of known methods as described above.

In yet another aspect, the invention solves the multiple disadvantages and problems of known treatments by providing safe, effective, integrative, holistic, whole-system methods and systems for treating acne and other inflammatory skin conditions that includes, inter alia, the steps of collecting and/or organizing and/or interpreting multiple patient data points, for example but not limited to, patient queries and/or patient histories and/or medical testing and/or observation of patient and/or examination of patient and/or evaluation of patient and/or conventional medicine and/or traditional medicine and/or complementary medicine and/or alternative medicine and/or causal factors of acne and/or causal factors of inflammatory skin conditions, determining relationships between multiple patient data points, applying relationships between multiple patient data, formulating treatment options, adjusting treatment options, structuring a treatment protocol, administering a treatment protocol, monitoring patient for response to administered treatment protocol, optionally reiterating one or more of these steps of collecting, organizing, interpreting, determining, applying, formulating, adjusting, structuring, administering, which novel methods and systems of the invention solve the multiple disadvantages and problems of known methods as described above, including reducing unwanted side-effects, restoring balance to the intestinal and/or skin microbiome, reducing and/or eliminating yeast overgrowth in the intestines, addressing and/or correcting ileocecal valve dysfunction and/or leaky gut issues, and reducing weight gain, reducing and/or eliminating nausea and abdominal pain and bloating.

In yet another aspect, the invention provides safe, effective, integrative, holistic, whole-system methods and systems for treating acne and other inflammatory skin conditions that includes, inter alia, the steps of collecting and/or organizing and/or interpreting multiple patient data points, for example but not limited to underlying causal factors of acne and other inflammatory skin conditions including those disclosed herein such as local chemical irritants, food toxins, environmental toxins, acne bacteria, acne bacteria enzymes, stress, touching of lesions, squeezing of lesions, poor digestion, immune dis-regulation, junk food diet, lack of pre-biotics, yeast overgrowth in the gut, leaky gut, wheat and/or gluten consumption, high-sugar food diet, high glycemic food diet, fried foods diet, altered microbiome, altered hormones, stimulants such as caffeine and theobromine, sugar consumption, high-temperature heated fats intake, consumption of milk and/or cheese food products, food allergens incompletely-digested foods, topical cutting oil, hormone disruptors, estrogen subtypes, progesterone, androgen, DHEA-S, imbalanced hormones, determining relationships between multiple patient data points, applying relationships between multiple patient data, formulating treatment options, adjusting treatment options, structuring a treatment protocol, administering a treatment protocol, monitoring patient for response to administered treatment protocol, optionally reiterating one or more of these steps of collecting, organizing, interpreting, determining, applying, formulating, adjusting, structuring, administering, and which novel methods and systems of the invention solved the multiple disadvantages and problems of known methods as described above, including reducing unwanted side-effects, restoring balance to the intestinal and/or skin microbiome, reducing and/or eliminating yeast overgrowth in the intestines, addressing and/or correcting ileocecal valve dysfunction and/or leaky gut issues, and reducing weight gain, reducing and/or eliminating nausea and abdominal pain and bloating and/or other inflammatory conditions in that when they are treated by providing safe, effective, integrative, holistic, whole-system methods and systems of the invention that includes, inter alia, the steps of collecting and/or organizing and/or interpreting multiple patient data points, for example but not limited to, patient queries and/or patient histories and/or medical testing and/or observation of patient and/or examination of patient and/or evaluation of patient and/or conventional medicine and/or traditional medicine and/or complementary medicine and/or alternative medicine and/or causal factors of acne and/or causal factors of inflammatory skin conditions, determining relationships between multiple patient data points, applying relationships between multiple patient data, formulating treatment options, adjusting treatment options, structuring a treatment protocol, administering a treatment protocol, monitoring patient for response to administered treatment protocol, optionally reiterating one or more of these steps of collecting, organizing, interpreting, determining, applying, formulating, adjusting, structuring, administering, such inflammatory conditions are improved at least for the reason that the underlying causal factors of the inflammation are also addressed.

In yet another aspect, the invention provides safe, effective, integrative, holistic, whole-system methods and systems for treating acne and other inflammatory skin conditions that includes, inter alia, the steps of collecting and/or organizing and/or interpreting multiple patient data points, for example but not limited to, patient queries and/or patient histories and/or medical testing and/or observation of patient and/or examination of patient and/or evaluation of patient conventional medicine and/or traditional medicine and/or complementary medicine and/or alternative medicine and/or causal factors of acne and/or causal factors of inflammatory skin conditions, determining relationships between multiple patient data points, for example but not limited to one or more of the following, viewing causal factors as a collection of environmental and/or individual-specific etiologic factors shared by acne and other inflammatory skin conditions, tailoring the novel methods and systems of the invention to the individual patient, selecting and implementing foundational steps to address these causal factors, conducting additional queries and/or assessments and/or medical testing, and which novel methods and systems of the invention solved the multiple disadvantages and problems of known methods as described above, including reducing unwanted side-effects, restoring balance to and/or reconfiguring the intestinal and/or skin microbiome, reducing and/or eliminating yeast overgrowth in the intestines, addressing and/or correcting ileocecal valve dysfunction and/or leaky gut issues, and reducing weight gain, reducing and/or eliminating nausea and abdominal pain and bloating and/or chronic fatigue and/or mental fogginess, as recognized and understood by one of ordinary skill in the art.

In yet another aspect, the invention provides safe, effective, integrative, holistic, whole-system methods and systems for treating acne and other inflammatory skin conditions that provides, inter alia, unexpected and positive synergies that are greater than a mere aggregation of individual treatment approaches, and in yet another aspect of the invention, such methods and systems provide such unexpected and positive synergies by operating cross-functionally in that they utilize multiple and/or different data inputs, including but not limited to, input from conventional medicine, as well as other forms of medicine, for example but not limited to, herbal, Ayurvedic, Chinese, homeopathic, functional, alternative, complementary, restorative, lifestyle, and other healing systems, and which novel methods and systems of the invention thereby solve the multiple disadvantages and problems of known methods as described above, including not approaching treatment in a piecemeal way, reducing unwanted side-effects, restoring balance to the perturbed intestinal and/or skin microbiome, reducing and/or eliminating yeast overgrowth in the intestines, addressing and/or correcting ileocecal valve dysfunction and backing up of colonic organisms into the small intestine and/or leaky gut issues, and reducing weight gain, reducing and/or eliminating nausea and abdominal pain and bloating.

In yet another aspect, the invention provides integrative, holistic, whole-system methods and systems for treating acne and other inflammatory skin conditions that provides, inter alia, multiple advantages readily recognized and understood by one of ordinary skill in the art based upon this disclosure, and provides solutions to the multiple disadvantages and problems of known methods as described above, which multiple advantages include, for example, but are not limited to, eliminating and/or greatly reducing the use of pharmaceuticals, many of which have negative side effects, eliminating and/or greatly reducing the use of topical medicaments and/or skin treatment compositions, which treat only skin surface symptoms, eliminating and/or greatly reducing the use of antibiotics, eliminating and/or greatly reducing the use of birth control pills, and thereby solving one or more of the multiple disadvantages and problems of known methods as described above.

In yet another aspect, the invention provides safe, effective, integrative, holistic, whole-system methods and systems for treating acne and other inflammatory skin conditions that provides, inter alia, multiple advantages readily recognized and understood by one of ordinary skill in the art based upon this disclosure, and provides solutions to the multiple disadvantages and problems of known methods as described above, which multiple advantages include, for example, but are not limited to, providing benefits and positive enhancements to the patient's physical and/or emotional well-being, for example but not limited to, by rebuilding and/or rebalancing the intestinal microbiome, by reducing and/or removing inflammation such as that resulting from molecular mimicry, by restoring the intestinal lining integrity, by modifying the epigenetic profile such to a normal pattern and/or functions more optimally, by reducing and/or eliminating pharmaceutical usage, by reducing and/or eliminating use of less safe and/or effective methods of treatment, by addressing the underlying causes that lead to acne and inflammatory skin conditions, by providing relief for some patients from: bloating, gastrointestinal pain, constipation, low energy, tiredness, chronic fatigue, mood swings, low emotional spirits, depression, yeast overgrowth, permeable intestinal lining, food allergies, headaches, sinus discomfort, and also by ameliorating and/or avoiding health issues by correcting imbalances and/or by correcting underlying health issues that are known to possibly develop into such conditions as diabetes, obesity, heart disease, irritable bowel syndrome, non-alcoholic fatty liver disease, autoimmune disease, allergies, decreased brain function, and also by reducing and/or eliminating overall inflammation in the body of the patient for at least the reasons that the underlying causal factors of acne and other inflammatory skin conditions are addressed, and also by repairing permeable intestinal lining issues, and by in particular for women of child-bearing age thereby reducing chances of transmitting allergic tendencies to the fetus and later the child, and also by eliminating unhelpful food in regards to acne and other inflammatory skin conditions, and also by clearing skin appearance thereby improving self-confidence of the patient and thereby, for example, improving employment eligibility status of the patient, and also by enhancing the social life of the patient, and thereby solving one or more of the multiple disadvantages and problems of known methods as described above.

In yet another aspect, the invention provides safe, effective, integrative, holistic, whole-system methods and systems for treating acne and other inflammatory skin conditions that provides, inter alia, multiple advantages readily recognized and understood by one of ordinary skill in the art based upon this disclosure, and provides solutions to the multiple disadvantages and problems of known methods as described above, which multiple advantages include, for example, but are not limited to, not approaching treatment in a piecemeal way, combining multiple knowledge inputs and/or patient data points for example but not limited to patient queries and/or patient histories and/or medical testing and/or observation of patient and/or examination of patient and/or evaluation of patient and/or conventional medicine and/or traditional medicine and/or complementary medicine and/or alternative medicine and/or causal factors of acne and/or causal factors of inflammatory skin conditions, and determining relationships between these multiple knowledge inputs, including knowledge inputs relating to cross-reactive inflammatory attacks and/or molecular mimicry, as further described herein, and/or patient data points for example but not limited to between the immune system and acne and other inflammatory skin conditions, which relationships are applied in formulating and/or adjusting treatment options, and then structuring and administering treatment protocols and then monitoring the patient for response to the administered treatment protocol, all in contrast to known treatment that may be subject to multiple disadvantages and problems, including being simplistic and/or non-inclusive and/or incomplete and/or ineffective and/or producing unwanted side effects, thereby solving one or more of the multiple disadvantages and problems of known methods as described above.

In yet another aspect, the invention provides safe, effective, integrative, holistic, whole-system methods and systems for treating acne and other inflammatory skin conditions that provides, inter alia, multiple advantages readily recognized and understood by one of ordinary skill in the art based upon this disclosure, and provides solutions to the multiple disadvantages and problems of known methods as described above, which multiple advantages include, for example, but are not limited to, addressing underlying causal factors for acne and other inflammatory skin conditions and thereby overcoming negative health issues such as gut dysbiosis, that is, imbalance and/or maladaptation on or inside the body, such as an impaired microbiota and, as a result such utilization of the inventive methods and systems described herein, overcoming negative health issues therein providing advantageous disease prevention aspects in that, by contrast to the known methods described above and the accompanying multiple disadvantages and problems of known methods, including, for example, using known treatment methods and the concomitant delayed evaluation and correction of causal factors, which often leads to other health problems, that is, co-morbidities, which co-morbidities are reduced and/or avoided by use of the inventive methods and systems described herein, for example but not limited to, providing integrative, holistic, whole-system methods and system that, inter alia, address underlying causal factors for acne and other inflammatory skin conditions, which reduces and/or eliminates delayed evaluation and correction of such causal factors, thereby overcoming negative health issues such as gut dysbiosis and thereby providing advantageous disease prevention aspects, and which reduces and/or eliminates overall inflammation in the body of the patient for at least the reasons that the underlying causal factors of acne and other inflammatory skin conditions are addressed, and thereby solving one or more of the multiple disadvantages and problems of known methods as described above.

In yet another aspect, the invention provides safe, effective, integrative, holistic, whole-system methods and systems for treating acne and other inflammatory skin conditions that provides, inter alia, multiple advantages readily recognized and understood by one of ordinary skill in the art based upon this disclosure, and provides solutions to the multiple disadvantages and problems of known methods as described above, which multiple advantages include, for example, but are not limited to, providing a reduction or elimination of collateral damage to the patient, as collateral damage is defined herein, including, whether it be physical, emotional and/or spiritual damage, such collateral damage resulting from utilization of known methods at least as described above, for example without limitation, collateral damage such as adverse reactions and/or allergic reactions caused by the medicines and/or drugs administered, adverse effect of even intermittent use of antibiotics on the normal intestinal flora that then favors overgrowth of eukaryotic organisms such as Candida albicans and which can lead to initial and even cycles of intestinal inflammation, leaky gut, and inflammatory attacks elsewhere in the body, and gastrointestinal disorders and/or autoimmune disorders; and collateral damage such as depression, prolonged dry skin, joint problems and/or nonalcoholic fatty liver resulting from use of 13-cis retinoic acid; and collateral damage such as Candida overgrowth and/or infertility resulting from prolonged use of birth control pills to treat acne; and collateral damage such as scarring resulting from mismanaged acne resulting from lack of understanding of the holistic approach of the invention disclosed herein; and collateral damage such as not properly addressing the causal factors of acne and other inflammatory skin conditions, as described herein, which then leads to collateral damage such as multiple disruptions for the patient whether physically, emotionally and/or spiritually, including inflammatory conditions and/or lack of ability of the patient to confront emotional issues underlying behaviors such as overconsumption of sugar and other unhealthy foods in excess.

In yet another aspect, the invention provides safe, effective, integrative, holistic, whole-system methods and systems for treating acne and other inflammatory skin conditions that provides, inter alia, multiple advantages readily recognized and understood by one of ordinary skill in the art based upon this disclosure, and provides solutions to the multiple disadvantages and problems of known methods as described above, which multiple advantages include, for example, but are not limited to, providing treatment options from which to select a treatment protocol that is supported by or modified by patient history iterative queries to collect, organize, interpret multiple patient data points and determining relationships between multiple patient data points and applying determined relationships by for example formulating treatment options from which to select a treatment protocol, and wherein data points include, for example and as describe and disclosed herein, events that enhance antigen exposure of the patient by increasing leaky gut for example via gluten, specific patient allergies, irritation of the gut by for example alcohol and/or non-steroidal anti-inflammatory drugs that is NSAIDs, patient exposure to toxins as described and disclosed herein, Candida yeast presence in the gut, and/or events that relate to the causal factors of acne and other inflammatory skin disorder, and wherein a dysregulating event such as, for example, viral infection, medication, and/or severe distress may be a concomitant causal factor that pushes inflammation where the other exposures alone did not, and whereby the invention provides safe, effective, integrative, holistic, whole-system methods and systems for treating acne and other inflammatory skin conditions wherein the patient is protected by local anti-inflammatories in the digestive tract such as, for example and as described and disclosed herein, demulcents, fiber, reduction of the diet fed yeast population, and support for the liver and/or other organs of elimination of toxins from the patient body, so as to substantially reduce and/or eliminate adverse reactions, and whereby, for example, Candida yeast is first reduced and controlled before glutamine is added to repair leaky gut to avoid the glutamine feeding the Candida yeast and fostering overgrowth and exacerbating the leaky gut if the gut immune system has not been first re-instructed to control Candida yeast presence at a lower level, and wherein afterwards hormonal levels are addressed, and wherein food allergy testing is conducted after the gut barrier has been repaired as described and disclosed herein to avoid false positive test results from large, recognizable food and/or microbial antigens passing through the leaky gut and testing positive for whatever the patient with leaky gut is eating, and wherein the sequence of addressing particular patient health issues is modified according to urgency of symptoms, such as perimenstrual acne flare, which requires starting specific treatments on initial visit, and/or addressing digestive issues first, as their clearing usually precedes acne and other inflammatory skin conditions being resolved, and/or addressing chronic fatigue that improves with resolution of digestive and chronic viral issues while implementing any treatment protocol with caution and careful monitoring and adjusting as needed in cases where the patient symptoms are exacerbated that results in part from over-aggressive treatment as recognized and understood by one of ordinary skill in the art based on the disclosure herein, and wherein the invention provides safe, effective, integrative, holistic, whole-system methods and systems for treating acne and other inflammatory skin conditions that provides the advantage of methods and systems of treatment that address the entire person, as the term “entire person” is herein defined, and as recognized and understood by one of ordinary skill in the art, and further meaning and including the inventive methods and systems address the patient physically, mentally, emotionally, spiritually and/or environmentally, and for example, if there are lifelong physical, mental, emotional, environmental and/or spiritual habits of the patient, which habits of the patient are often supported by family, peers, community, and/or society, then these habits of the patient are addressed as part of addressing the entire person, and addressed at multiple levels to insure success, and for example and without limitation by addressing any environmental factors like exposure to mold and/or toxins, and whereby the inventive methods and systems of treatment disclosed herein provide these considerations are nested in an axis in a different, interactive dimension from the original plane of the plane of the basic treatment module.

### BRIEF DESCRIPTION OF THE DRAWINGS

The detailed description and exemplary embodiments may refer to the accompanying drawings and figures, which are disclosed as examples and not intended to be limiting, and in which other characteristics and advantages of the invention are recognized and understood by one of ordinary skill in the art. Further objects, features, aspects and advantages of the embodiments of this disclosure become apparent to and recognized and understood by one of ordinary skill in the art upon reference to the drawings and the detailed description herein including the claims. Note that the disclosure herein will readily be recognized and understood by one of ordinary skill in the art to have broad usefulness and application and while some embodiments are disclosed for illustrative purposes in providing a full and enabling disclosure of the present invention, some embodiments, such as adaptations, variations, modifications, and equivalent arrangements, are implicitly disclosed by the embodiments described herein and fall within the scope of the invention. Accordingly, the disclosure herein is illustrative and exemplary of the invention, and is made for the purposes of providing a full and enabling disclosure of the invention.

FIG. 1 is an illustration of a chart of exemplary steps of the invention in accordance with an embodiment of the invention regarding methods and systems for treating acne and other inflammatory skin conditions.

FIG. 2 is an illustration of a chart of exemplary steps of the invention in accordance with another embodiment of the invention regarding methods and systems for treating acne inflammation and dermatitis and other inflammatory skin conditions.

FIG. 3 is an illustration of a chart of exemplary steps of the invention in accordance with yet another embodiment of the invention regarding methods and systems for treating acne inflammation and cystic acne and other inflammatory skin conditions.

### DETAILED DESCRIPTION

Embodiments of the invention are described and disclosed in detail below and the features and/or aspects and/or elements described and disclosed in detail with respect to certain illustrative example embodiments may be combined and sub-combined in and/or with various other illustrative example embodiments as recognized and understood by one of ordinary skill in the art. Some embodiments, whether individually and/or collectively, may be components of a larger system, wherein certain steps may take precedence over and/or otherwise modify their application as recognized and understood by one of ordinary skill in the art. This detailed description and disclosure of objects, features, aspects and advantages of the invention disclosed herein are readily recognized and understood by one of ordinary skill in the art.

Causal factors of acne and/or acne inflammation includes, but is not limited to the following items that are interrelated and lead to acne and/or acne inflammation, for example, local chemical irritation and/or food toxins and/or environmental toxins that relates to decreased activity in Treg cells, that is, regulatory T cells that are a subpopulation of T cells that suppress immune response and help to maintain homeostasis and self-tolerance and preventing autoimmunity, at least by inhibiting T cell proliferation and cytokine production, and the decreased activity in Treg cells that relates to increased inflammation that relates to acne inflammation; acne bacteria that relates to enzymatic conversion of fats into irritating free fatty acids that relates to inflammation and/or plugging and/or blockage of duct openings that relates to acne inflammation; acne bacteria that acts as immune adjuvants for food-derived molecules and/or microbial antigens and/or toxins such as lipopolysaccharides such as from gram negative bacteria of the colon, all of which relate to acne inflammation; stress that relates to acne inflammation and relates to touching and/or squeezing of lesions and relates to poor digestion and relates to immune dis-regulation that all relate to acne inflammation; stress that relates to ingesting so-called “junk foods”, which term means, as recognized and understood by one of ordinary skill in the art and as defined herein, and also includes meaning “junk food diet”, that is, a diet of foods such as, for example, pizza, deep fried foods, potato chips, soda, chocolate that relate to lack of pre-biotics that relates to pro-inflammatory bacteria in the intestines that relates to acne inflammation and the lack of pre-biotics, probiotics and fiber also relates to yeast growth in the intestines that relates to leaky gut that relates to acne inflammation; junk foods that relate to advanced glycation end products that lead to altered fats and lead to cross-reactive attacks as described herein, including for example without limitation, enteric organisms in and/or on the skin that may serve as cross-reactive targets to multiple organs and/or tissues within the body; gluten in foods that relates to zonulin that relates to leaky gut and food allergen exposure that relates to acne and other inflammatory skin conditions; high-sugar food consumption that relates to yeast growth in the intestines that relates to inflammation of the intestinal lining that relates to leaky gut that relates to acne inflammation for example without limitation to inflammatory attack of Malassezia yeast in the follicle that leads to acne inflammation; high sugar food consumption that relates to increased insulin that relates to increased insulin growth factor-1 that relates to acne inflammation for example without limitation increased insulin growth factor-1 that stimulates the protein complex known as mammalian target of rapamycin complex 1 that is mechanistic target of rapamycin complex 1 that is mTORC1 that stimulates sebum production; incompletely-digested foods especially milk-based foods that relate to leaky gut and relate to leakage into the lymphatics and/or the blood supply for the small intestines and relate to cross reactions and immune attacks on sebaceous and follicular structures that relates to acne inflammation; wheat and wheat-based food consumption that relates to zonulin and/or zonulin family protein release that relates to leaky gut that relates to acne inflammation; consumption of stimulants such as coffee that relates to altered hormone balance and/or adrenal depletion and/or altered microbiome and acne inflammation; consumption of fried foods and sugars and high temperature-heated fats that relates to increased advanced glycosylated end products that for example without limitation affects sebaceous secretions that relates to inflammation and relates to acne inflammation; consumption of fried foods and sugars and high temperature-heated fats that relates to poor digestion that for example without limitation alters dietary food molecules that relates to acne inflammation and relates to leaky gut that relates to acne inflammation; consumption of food allergens and/or milk and/or milk products and/or cheese that relate to cross-reactive food allergens, insulin growth factor-1, hormones that relate to poor digestion and leaky gut that relates to acne inflammation; hormone disruptors and/or contact with topical cutting oil and/or imbalanced hormones and/or estrogen subtypes and/or progesterone and/or androgen and/or dehydroepiandrosterone sulfate that relates to acne inflammation.

Some embodiments of the methods and systems of the invention include iterations of one or more of the steps of the invention, wherein the iterations of steps of the invention in view of causal factors of acne and other inflammatory skin conditions include for example but not limited to iterating the steps of: collecting multiple patient data points; organizing multiple data points; interpreting multiple data points; determining relationships between the multiple patient data points; applying relationships between the multiple patient data points by formulating treatment options based on relationships between the multiple patient data points and optionally adjusting treatment options based on relationships between the multiple patient data points; structuring a treatment protocol based on treatment options; administering treatment protocol to a patient; and monitoring patient response to treatment protocol, and wherein some embodiments of the methods and systems of the invention include optionally adjusting treatment options is based on temporally sequential relationships between the multiple patient data points.

A detailed description of causal factors of acne and/or acne inflammation disclosed herein lists items that are, for example without limitation, at least part of input or iterative input in some embodiments of the methods and systems of the invention, wherein “input” or “iterative input”, as recognized and understood by one of ordinary skill in the art, also includes meaning, as appropriate given the context of the usage, for example without limitation, input or iterative input from multiple patient data points, input or iterative input relationships between multiple patient data points, input or iterative input from one or more patient queries, input or iterative input from patient history, input or iterative input from medical testing, input or iterative input from observation of the patient, input or iterative input from examination of the patient, input or iterative input from evaluation of the patient, input or iterative input from conventional medicine, input or iterative input from traditional medicine, input or iterative input from complementary medicine, input or iterative input from alternative medicine, input or iterative input from causal factors of acne, and input or iterative input from causal factors of inflammatory skin conditions.

Some embodiments of the methods and systems of the invention include data points and/or input and/or iterative input, wherein the terms data points and input and iterative input, as recognized and understood by one of ordinary skill in the art, also includes meaning, as appropriate given the context of the usage, for example without limitation, data points and/or input and/or iterative input from an understanding of and/or knowledge of: various medicines and/or various healing systems as described herein, such as without limitation, conventional medicine, traditional medicine, complementary medicine, alternative medicine, functional medicine, restorative medicine, precision medicine, causal factors of acne and/or acne inflammation, reconsideration of causal factors of acne and/or acne inflammation, and said input or iterative input, as recognized and understood by one of ordinary skill in the art, also includes meaning, as appropriate given the context of the usage, for example without limitation, input or iterative input from: an understanding of and/or knowledge of immune recognition as recognized and understood by one of ordinary skill in the art and as defined herein; an understanding of and/or knowledge of cross-reactive immune recognition as recognized and understood by one of ordinary skill in the art and as defined herein; an understanding of and/or knowledge of liver functionality such as a filter and processing factory as recognized and understood by one of ordinary skill in the art and as defined herein including an understanding of and/or knowledge of cytochromes P450 family of enzymes and the solubilization of molecules and/or making more reactive molecules so that they can be chemically coupled to a transporter for removal from the body; an understanding of and/or knowledge of stuffing emotions as recognized and understood by one of ordinary skill in the art and as defined herein; an understanding of and/or knowledge of gut permeability that is leaky gut as recognized and understood by one of ordinary skill in the art and as defined herein; an understanding of and/or knowledge of immune tolerance as recognized and understood by one of ordinary skill in the art and as defined herein; an understanding of and/or knowledge of Candida that is yeast as recognized and understood by one of ordinary skill in the art and as defined herein; an understanding of and/or knowledge of seborrheic dermatitis that is seborrhea as recognized and understood by one of ordinary skill in the art and as defined herein; an understanding of and/or knowledge of the peptide bradykinin and inflammation; an understanding of and/or knowledge of multiple information and/or multiple inputs relating seborrheic dermatitis, dermatographic urticaria, herbal treatments, medical practitioner insight and medical intuition, fuzzy logic, relationships by and between mental, emotional, spiritual issues and/or states, acne, inflammatory skin conditions, inflammation, meditation, healing forces, dietary supplements, nutrients, herbs, herbal treatments, physical and emotional and spiritual inner workings, the biochemistry of skin aging, as recognized and understood by one of ordinary skill in the art and as disclosed herein; an understanding of and/or knowledge of human leukocyte antigen, that is HLA, system or complex in humans, and which is involved in the individual specificity of the immune recognition and reaction in which antigens are presented to the immune system by self HLA structures and are recognized as unique in conjunction with those molecules.

that is that group of related proteins encoded by the major histocompatibility complex gene complex in humans and which cell-surface proteins are implicated in regulation of the immune system, as well as an understanding of and/or knowledge of the HLA-individualized nature of cross-reactive immunity to food and microorganisms as related to the causal factors of acne and other inflammatory skin conditions and inflammation throughout the body, and for example and without intending to be limiting, in an embodiment of the invention, multiple patient data points and/or input and/or iterative input were collected, organized, interpreted, processed, structured and/or related and relationships were determined between multiple patient data points and relationships between multiple patient data points were applied by formulating treatment options and adjusting treatment options for example in view of the relationships between multiple patient data points and structuring a treatment protocol from the treatment options and administering the treatment protocol to the patient and monitoring the patient for response to the administered treatment protocol, and the steps of collecting organizing, interpreting, processing, structuring, relating, and applying relationships between multiple patient data points and formulating treatment options and structuring a treatment protocol from the treatment options and administering the treatment protocol to the patient and monitoring the patient for response to the administered treatment protocol included, for example and as further described and disclosed herein, reducing metabolites that feed the yeast population including for example Candida, and/or pro-inflammatory intestinal bacteria by for example an elimination diet wherein eliminating from the diet for example foods that contain and/or are made with yeast and/or yeast byproducts reduces the tolerizing effects of these byproducts on the gut immune system, allowing the gut immune system to control yeast populations at lower level, and thereby reducing the yeast population so that it is less likely a large resistant population will remain after administering the treatment protocol, and in some cases adjusting the treatment protocol where the patient experienced a large amount of yeast overgrowth and/or intestinal and/or other hypersensitivity symptoms and upon treatment with for example anti-yeast supplements and/or herbs and/or drugs subsequently experience a so-called “die-off” or Herxheimer-like reaction as recognized and understood by one of ordinary skill in the art, and afterwards adjusting the treatment protocol by providing a way to improve removal of toxins from the body, for example with administration of a fiber product, soluble and insoluble, such as for example a psyllium or flax seed or chia seed product or combination product with water to move digestive contents more rapidly through the digestive tract, and to bind out toxins as yeast and/or harmful bacteria, and to provide a fiber matrix to support a more diverse, healthier microbial population to ecologically reduce the population of yeast, and wherein treatment options are adjusted when the patient experiences constipation by administering progressively more vigor, including increased fiber and water, high dose vitamin C, and followed by Triphala and aloe to restore normal bowel regularity, which involves applying understanding of and/or knowledge of relationships as described and disclosed in the inventive methods and systems of the invention, wherein the understanding of and/or knowledge of relationships includes for example that the toxic metabolites such as Indole that are absorbed from the stool during a state of constipation and which toxic metabolites collectively enter the blood stream and/or lymphatics and contribute to acne and other inflammatory skin conditions and to inflammation, and an understanding of and/or knowledge of the constipation backing up stool into the small intestines causing fecal bacteria to be pushed into the small intestines, and when the patient has a leaky gut, such bacteria enters both the surrounding lymphatics and blood stream, and wherein such bacteria are delivered throughout the body and/or skin organelles of excretion such as for example the sebaceous glands leading to acne and/or inflammatory skin conditions and/or inflammation throughout the body, and further including in the treatment protocol teaching patients to massage the ileocecal valve and avoid foods such as gritty foods for example, popcorn, poorly-chewed nuts and/or raw vegetables, and especially carrots, which physically irritate the ileocecal valve and for example cause it to hang open and/or not completely closed, and hot and spicy foods such as cayenne pepper, and all of these that favor the valve hanging open then allow backflow from the colon to the small intestines, and in an embodiment of the invention, for example without intending to be limiting: administering supplements such as, for example without intending to be limiting, quercetin to calm the intestinal tract and/or liver-supporting herbal combinations as described and disclosed herein; and/or administering digestive enzymes to reduce allergenicity of food before administering anti-yeast supplements such as for example caprylic acid; and/or reducing sugar form the diet to reduce release of insulin and/or insulin like growth factor that is IGF-1 that leads to acne and other inflammatory skin conditions for example without limitation by increasing testosterone sensitivity and leading to increased lipid production by sebaceous glands thereby predisposing the glands to acne and inflammation; administering for example a berberine, baicalin, isatis root combination to kill not only yeast, but also other pathologic bacteria in the intestinal tract without destroying normal gut bacteria which gut bacteria destruction is a common negative and unwanted side effective of antibiotics, and furthermore, if that is unsuccessful in improving the patient's health, then the stool tests are utilized to identify resistant yeast species and/or pathologic bacteria that might require the temporary and limited antibiotic treatment such as for example, nystatin, to control yeast overgrowth; reducing and/or eliminating gluten to reduce the level of zonulin being released, as zonulin opens the intercellular spaces between the epithelial lining cells in the small intestine causing leaky gut; monitoring each administration of each step of the methods and systems of the invention to determine whether adjustment is needed, for example without limitation, wherein the goal is to reduced and/or eliminate any die-off reaction upon commencing with an anti-yeast product, and wherein the adjustment needed is focused the steps before and the steps after adding an anti-yeast product to reduce inflammation of the gut lining, the leakage across that lining, and the pressure on the liver to break down allergens and toxins in the blood flow from the digestive system that it filters; administering probiotics at this point probiotics for example without limitation Acidophilus and/or Bifidobacteria to repopulate normal flora in the digestive system, and administering prebiotics for example as food or supplements to favor the growth of healthy bacteria in the gut wherein such healthy bacteria crowd out yeast and/or harmful bacteria; administering, once yeast population is under control as described and disclosed herein, an assessment of adherence by the patient to the treatment protocol and thereafter addressing symptoms of bloating, gas, constipation and/or diarrhea by administering products as described and disclosed herein to retire gut integrity of the gut lining and repair leaky gut, including for example without limitation intestinal emollients, such as deglycyrrhizinated licorice that is DGL and/or aloe and/or marshmallow root, that protects the gut lining, and wherein an ingredient for healing the leaky gut is glutamine that restores integrity of the gut lining, however administering with an understanding and/or knowledge of the relationship of glutamine and glucose such that for some patients not only is the yeast population under control but also that the gut immune system be in a state of yeast reduction long enough to reduce and reset the control of the yeast population at a lower level, so as to avoid continued feeding of the yeast and paradoxically experiencing leaky gut and worsening the acne and/or other inflammatory skin conditions and/or inflammation; administering vitamins such as for example without limitation vitamin A to support immune function and reduce excess keratotic plugging as when applied topically as retinoic acid, and/or vitamin D-3 that enhances immune function and protects against autoimmune disorders, and/or vitamin C as an antioxidant.

Some embodiments of the methods and systems of the invention include input or iterative input, wherein input or iterative input, as recognized and understood by one of ordinary skill in the art, also includes meaning, as appropriate given the context of the usage, for example without limitation, input or iterative input from an understanding of and/or knowledge of immune recognition as recognized and understood by one of ordinary skill in the art and as defined herein and/or an understanding of and/or knowledge of cross-reactive immune recognition as recognized and understood by one of ordinary skill in the art and as defined herein, and for both also includes meaning an understanding that and/or knowledge that T-lymphocyte that is white blood cell recognition of antigens such as foreign microbes and/or foreign harmful substances and/or threats from within such as tumors and/or infected cells, and the initial T-lymphocytes, after recognition, dividing to create multiple T-lymphocytes with the same antigen recognition receptor that is a large number of T-lymphocytes with the same antigen recognition receptor that search for a similar antigen; however, when parts of skin cells and/or altered skin cells have a similar appearance to antigens, for example without limitation by molecular mimicry, can be recognized as an antigen by T-lymphocytes, which is known as cross-reaction, and when the cross-reactive substance is part of the body, then the result may be inflammation from an auto-immune attack, such that T-lymphocytes may attack parts of skin cells and/or altered skin cells having similar appearance to antigens and/or other organs as a result of molecular mimicry.

Some embodiments of the methods and systems of the invention include data points and/or input and/or iterative input, wherein the terms data points and input and iterative input, as recognized and understood by one of ordinary skill in the art, also includes meaning, as appropriate given the context of the usage, for example without limitation, data points and/or input and/or iterative input from an understanding of and/or knowledge of liver functionality such as a filter and processing factory as recognized and understood by one of ordinary skill in the art and as defined herein and also includes meaning that the liver filters the blood and removes chemicals, toxins, microbes and other materials as understood by one of ordinary skill in the art and as defined herein, as well as that the liver processes such chemicals, toxins, microbes and other materials that have been removed from the blood and prepares them for export that is removal that is expelling them from the body, for example without limitation by, chemically altering them such as when such chemicals, toxins, microbes and other materials are coupled to carrier molecules, and then the exporting, removal, expelling is by way of, for example without limitation, the kidneys, bile, and/or stool, and further, in cases where the liver processes for removal fat toxins, as understood by one of ordinary skill in the art, wherein the fat toxins are made soluble to be excreted, and they are chemically altered, for example without limitation, by oxidation to become water soluble and/or are bound to bile for elimination from the body via the stool.

Some embodiments of the methods and systems of the invention include data points and/or input and/or iterative input, wherein the terms data points and input and iterative input, as recognized and understood by one of ordinary skill in the art, also includes meaning, as appropriate given the context of the usage, for example without limitation, data points and/or input and/or iterative input from an understanding of and/or knowledge of stuffing emotions as recognized and understood by one of ordinary skill in the art and as defined herein and also includes meaning when certain for example without limitation overwhelming painful emotions for example without limitation anger and/or fear and/or feelings of discomfort such as without limitation self-loathing, guilt, embarrassment, loss, worry, lead to a behavior wherein so-called comfort food, as recognized and understood by one of ordinary skill in the art, is consumed and/or other addictive behaviors are adopted to cover over such certain painful emotions, and which behaviors need to be addressed prior to or along with restoration of nutritional balance, and also includes meaning an understanding of and/or knowledge of inter-relationships between food, digestion and the skin.

Some embodiments of the methods and systems of the invention include data points and/or input and/or iterative input, wherein the terms data points and input and iterative input, as recognized and understood by one of ordinary skill in the art, also includes meaning, as appropriate given the context of the usage, for example without limitation, data points and/or input and/or iterative input from an understanding of and/or knowledge of gut permeability that is leaky gut as recognized and understood by one of ordinary skill in the art and as defined herein and also includes meaning that in the body, one of the largest sources of foreign harmful substances, as disclosed herein, is located in the digestive tract and the flora on which it feeds, and also includes meaning that with normal digestion any material entering the bloodstream is in the form of amino acids and/or small peptides, but with poor and/or incomplete digestion and/or leaky membranes for example without limitation leaky membranes of the digestive tract that is leaky gut, and including leaky membranes on the border of the small intestines, there are larger peptides and/or toxins that escape into the bloodstream and/or the lymphatics, thereby acting as targets for inflammation and/or targets and/or stimuli for cross-reaction, as defined herein, any place in the body and/or skin where such larger peptides and/or toxins are delivered, for example without limitation, by the bloodstream and/or the lymphatics.

Some embodiments of the methods and systems of the invention include data points and/or input and/or iterative input, wherein the terms data points and input and iterative input, as recognized and understood by one of ordinary skill in the art, also includes meaning, as appropriate given the context of the usage, for example without limitation, data points and/or input and/or iterative input from an understanding of and/or knowledge of immune tolerance as recognized and understood by one of ordinary skill in the art and as defined herein and also includes meaning that good immune tolerance is for example without limitation when your body does not react against your own cells, body chemicals, flora such as without limitation skin cells, certain yeast and bacteria, organs like the kidneys, and also includes meaning that bad immune tolerance is for example without limitation, tolerance to tumors, pathogens, and/or intercellular pathogens such as when recognized by the cell surfaces of virally-infected cells, and/or when yeast population increases in the gut such that over time the gut and/or gut immune system, as disclosed herein, becomes more tolerant toward the yeast population, and also includes meaning that for example without limitation after a period of time of at least one or more of a low sugar diet, low-yeast diet, anti-yeast supplements, probiotics, the yeast population decreases and the immune system tolerance adjusts to that is allowing a small yeast population, and also includes meaning that for example without limitation bad immune tolerance may occur after consuming large amounts of sugar and/or a little yeast and then the good immune tolerance is broken resulting in bad immune tolerance; and data points and/or input and/or iterative input from an understanding of and/or knowledge of yeast and/or fungi byproducts including for example the proteins they produce that can tolerize the gut immune system to Candida and/or other yeasts leading to a Candida and/or yeast overgrowth and leading to aggravation of cross-reactive attacks, for example as a result of yeast and/or fungi being eukaryotes and the relation to a protein transporter human sortilin that is the mammalian orthologue sortilin that transports proteins from the Golgi where it is activated by furin-mediated cleavage of its pro-domain that is similar to the furin-mediated cleavage required to activate the spike protein of SARS-CoV-2, as recognized and understood by one of ordinary skill in the art, and that yeast and/or fungi and their byproducts sensitize the immune to a variety of organs, which then become potential cross-reactive targets and lead to inflammation and/or inflammatory skin conditions and/or acne, and the novel methods and systems of the invention includes an understanding and/or knowledge of relationships of dietary yeast and Candida yeast and cross-reactivity causing gut inflammation and/or cross-reactivity of yeast, for example without limitation, in the follicles, and milk sensitivity as a causal factor of acne and/or inflammatory skin conditions and/or inflammation and relationships to hormones, IGF-1 activation, and other components in milk and cross-reactivity of the sebaceous glands and/or PFU; and some embodiments of the methods and systems of the invention include utilizing the steps described herein to provide safe, effective, integrative, holistic, whole-system methods and systems for treating acne and other inflammatory skin conditions and include providing adrenal support for patients worn out from chronic allergy and/or inflammation, including inflammatory skin conditions, and in some embodiments of the methods and systems of the invention, patients with multiple allergies, abnormal cortisol curves, and/or chronic fatigue, who regress with increased stress and perk up temporarily on corticosteroids and have weak gracillus, thigh adductor and/or gastrocnemius muscles are provided more rest and certain supplements, and the adrenal support includes for example raw adrenal gland extract, Ashwagandha, B complex vitamins, ginseng, Siberian ginseng, Eleutherococcus senticosus, gotu kola, Pantothenic acid, Tyrosine, antioxidants, super oxide dismutase, that is SOD, and elements supporting its synthesis including manganese, selenium; Vitamin E, Vitamin C, bioflavonoids.

Some embodiments of the methods and systems of the invention include data points and/or input and/or iterative input, wherein the terms data points and input and iterative input, as recognized and understood by one of ordinary skill in the art, also includes meaning, as appropriate given the context of the usage, for example without limitation, data points and/or input and/or iterative input from an understanding of and/or knowledge of Candida, that is, yeast, as recognized and understood by one of ordinary skill in the art and as defined herein and also includes meaning that while Candida is a most common yeast present in the digestive that is the intestinal tract and is considered normal, as recognized and understood by one of ordinary skill in the art and as defined herein, when it does not overpopulate such that the Candida increase greatly in population in relation to the healthy bacteria present in the digestive tract that is the intestinal tract, and the Candida becomes problematic for the remaining bacteria, as recognized and understood by one of ordinary skill in the art and as defined herein, and for example without limitation, after taking antibiotics healthy bacteria present in the digestive that is the intestinal tract are killed but not the Candida, which then reproduce to overpopulate, and which overpopulation of Candida is supported by a diet, that is, often a Western diet, as recognized and understood by one of ordinary skill in the art and as defined herein, including simple starches and sugars that operate as aggravators favoring Candida growth, and thereby resulting in the immune system attacking Candida, that is, yeast or yeast such as Malassezia yeast that is commonly present in and/or on the skin and/or skin-related tissues such as for example without limitation a hair follicle, as recognized and understood by one of ordinary skill in the art and as defined herein, causing for example without limitation, inflammation and/or inflammatory skin conditions, including seborrhea that leads to seborrheic dermatitis, as recognized and understood by one of ordinary skill in the art and as defined herein, and also an understanding of and/or knowledge of an abnormal gut microbiome leading to leakage of gut contents into the small intestine for example without limitation as a result of constipation and/or malfunction of the valve between the ilium and the cecum for example resulting from mechanical and/or caustic irritation and/or poor valve integrity that is small intestinal bacterial overgrowth that is SIBO in which for example fecal bacteria translocated into the small intestines which is complicated by leaky gut and wherein gram negative bacteria and their toxins to enter the lymphatics and/or blood stream and/or the blood supply for the small intestines and resulting in, for example without limitation, acne and/or other inflammatory skin conditions.

Some embodiments of the methods and systems of the invention include data points and/or input and/or iterative input, wherein the terms data points and input and iterative input, as recognized and understood by one of ordinary skill in the art, also includes meaning, as appropriate given the context of the usage, for example without limitation, data points and/or input and/or iterative input from an understanding of and/or knowledge of Candida as a eukaryotic organisms having many biochemical similarities to human systems and capable of influencing immune responses in multiple directions at the same time, for example without being limiting, by an ability to induce inflammation via cross-reactive attacks against the skin commensal, Malassezia, formerly Pityrosporum, its many amino acids and secreted factors, and/or a variety of human organs including thyroid, placenta, ovary, adrenal, thymus, liver, pancreas, spleen, brain and kidney, wherein many of its proteins share homology to human proteins, and thereby certain of its antigens can induce high dose tolerance to allow its persistence, as well as inflammation causing disruption of the barrier layer, simultaneously in the digestive tract, and both of which can be enhanced by cross-reactive antigens from other yeasts and fungi. Some embodiments of the methods and systems of the invention include multiple data points and/or input and/or iterative input from an understanding of and/or knowledge of reducing excess simple carbohydrates in the diet to prevent feeding these yeasts, as well as reducing yeast extracts and byproducts in foods made by other yeasts such as Saccharomyces in wine, beer, bread, and vinegar, all of which favor overgrowth of yeast in the gut and subsequently lead to leaky gut and inflammatory skin conditions such as acne, wherein antigens from the yeasts escape the intestinal lumen to the surrounding lymphoid tissue leading to cross-reactive sensitization against host tissues, such as is found in the PFU, leading to acne. Some embodiments of the methods and systems of the invention include, as disclosed herein, prebiotics and/or probiotics and/or natural anti-yeast herbs and/or anti-yeast supplements, fiber, wherein the yeast population is shifted by favoring more diversity of competing intestinal flora as well as a gut immune response to prevent yeast overgrowth, and wherein limiting and/or eliminating the migration of antigens from the yeast into the intestines reduces and/or eliminates inflammation and/or leaky gut, including for example limiting and/or eliminating inflammation through antigen movement into the surrounding lymphatic tissue that is Peyer's patches, and/or limiting and/or eliminating SIBO by limiting and/or eliminating passive migration through an incompetent open ileocecal valve.

Some embodiments of the methods and systems of the invention include data points and/or input and/or iterative input, wherein the terms data points and input and iterative input, as recognized and understood by one of ordinary skill in the art, also includes meaning, as appropriate given the context of the usage, for example without limitation, data points and/or input and/or iterative input from an understanding of and/or knowledge of dermatographic urticaria that is dermatographism that is urticaria that is chronic idiopathic urticaria that is hives, all of these terms as recognized and understood by one of ordinary skill in the art and as defined herein, and also includes meaning an understanding of and/or knowledge of the adverse response by patients, either physically and/or emotionally, to medicinal treatments such as, without limitation, antihistamines, H-1 blockers that is medications that block the action of histamine at the H-1 receptor, and H-2 blockers that sometimes are added to control urticaria and/or o better block histamine responses, and also includes meaning an understanding of and/or knowledge of relationships between inflammatory skin conditions and/or inflammatory reactions in the skin, basophils that release histamines, and autoimmune triggers such as autoantibodies directed at skin mast cell receptors, and also includes meaning an understanding and/or knowledge as disclosed in the example herein relating to chronic intermittent urticaria.

Some embodiments of the methods and systems of the invention include data points and/or input and/or iterative input, wherein the terms data points and input and iterative input, as recognized and understood by one of ordinary skill in the art, also includes meaning, as appropriate given the context of the usage, for example without limitation, data points and/or input and/or iterative input from an understanding of and/or knowledge of dietary supplements, nutrients, herbs, herbal treatments, as recognized and understood by one of ordinary skill in the art and as defined herein and also includes meaning the following whether utilized alone and/or in combinations as disclosed herein, for example and without intending to be limiting, soy extracts, resveratrol, green tea, curcumin, aloe vera, chamomile, witch hazel, chickweed, plantain, jewelweed, oats, berberine, essential oils such as tea tree and oregano oil, stinging nettle, calendula; fiber sources such as psillium husks, chia seed, Acacia fiver, flax soaked first in water, quercetin, silimarin, artichoke extract, curcumin, dandelion root, betaine hydrochloride with pepsin, pancreatic enzymes, digestive enzymes such as amylase, dipeptide peptidase that is DPP-IV, capryllic acid, bifidobacteria and lactobacillus combination, berberine, coptis, Scutellaria bicailensis, fructo-oligo saccharides, Jerusalem artichoke, zinc picolinate, vitamin B-6 in the form of pyridoxal-5-phosphate, glutamine and mucoid herbs, chromium chelates, fenugreek seed extract, niacin, inositol, burdock root and seed such as Arctium lappa, Cleavers such as Galium aparine, bromelain, calcium-D-gluacarate, Chaste tree such as Vitex castus agnes, vitamin A, vitamin C, vitamin D-3 such as vitamin D-3 at 5000 units per day dosage, bentonite clay, Gotu kola, chickweed ointment, stinging nettle, yellow dock such as Rumex crispus, fish oil such as those rich in omega-3 such as docohexaenoic acid (“DHA”) and eicosapentaenoic acid (“EPA”) and/or omega-6, which omega-3 and omega-6 are precursors of anti-inflammatory prostaglandins, and such fish oil and fish oil from salmon and/or mackerel and/or trout, zinc carnosine, demulcents, essential fatty acids such as found in starflower such as Borago officinalis that is Borage and/or evening primrose oil and/or flaxseed oil, utilizing gritty soap, wet dressings as when severe eczema patients place wet dressings and/or wet pajamas with waterproof covering to keep their skin moist, oligoproanthocyanadins (“OPC”) citrus bioflavonoids, rutin, reservatrol, calcium, magnesium, potassium carbonates, bicarbonates, Arnica montana.

Some embodiments of the methods and systems of the invention include data points and/or input and/or iterative input, wherein the terms data points and input and iterative input, as recognized and understood by one of ordinary skill in the art, also includes meaning, as appropriate given the context of the usage, for example without limitation, data points and/or input and/or iterative input from an understanding of and/or knowledge of making sense and/or using information and/or input from the totality of evidence surrounding a situation, and/or practitioner “insight”, and/or “medical intuition”, which terms “insight” and “medical intuition” mean, as recognized and understood by one of ordinary skill in the art, also includes meaning, for example without intent to limit, understanding and/or knowledge and/or information that is known intuitively by a practitioner and/or medical professional, and which includes an understanding of and/or knowledge of integrating and/or internalizing multiple types of data and/or data points, and which includes an understanding of and/or knowledge of utilizing so-called fuzzy logic, as recognized and understood by one of ordinary skill in the art and as defined herein, to lead to and to create a more focused picture of a situation from incomplete data, and which includes an understanding of and/or knowledge of input, or an understanding of and/or knowledge of iterative inputs, as in some embodiments of the methods and systems of the invention, that provide a broad pattern of information and/or data points that guide a practitioner and/or medical professional to an individual-specific organization, interpretation, determination of relationships between multiple patient data points and/or relationships between input and/or iterative inputs, application of such relationships, treatment adjustments, treatment protocol administration, as found in some embodiments of the methods and systems of the invention; and an understanding of and/or knowledge of, for example without limitation, inter-relationships between mental, emotional, spiritual issues, acne, inflammatory skin conditions, inflammation, diseases, meditation, therapy, support groups, spiritual healing, psychic healers, prayer healers, shamans, healing forces as well as an understanding of and/or knowledge of zoopharmacognosy that is animals appearing to know what they need to be healed when they are in need of healing by ingesting certain compounds that are found in roots, seeds, leaves, minerals, and relating this understanding and/or this knowledge in some embodiments of the methods and systems at multiple steps of the invention.

Some embodiments of the methods and systems of the invention include data points and/or input and/or iterative input, wherein the terms data points and input and iterative input, as recognized and understood by one of ordinary skill in the art, also includes meaning, as appropriate given the context of the usage, for example without limitation, data points and/or input and/or iterative input from an understanding of and/or knowledge of skin and/or acne and/or inflammatory skin conditions, as a reflection of the whole being and/or a reflection of your inner workings, including physical and emotional and spiritual workings, as recognized and understood by one of ordinary skill in the art and as defined herein, the biochemistry of skin aging and including for example without limitation nutrition, using sunscreen, cessation of smoking, taking appropriate dietary supplements and nutrients, such as without limitation, vitamin C, vitamin E, mixed carotenoids such as in carrots and other orange, yellow or red fruits and vegetables, and free radical quenchers and antioxidants including for example without limitation proanthocyanidins, bioflavonoids such as without limitation found in purple skin of grapes, grape seeds, and Polypodium leucotmos such as found in Central America, citrus fruits, green tea for example without limitation containing epigallocatechin gallate, selenium salts, N-acetyl-Cysteine, lipoic acid.

Some embodiments of the methods and systems of the invention include data points and/or input and/or iterative input, wherein the terms data points and input and iterative input, as recognized and understood by one of ordinary skill in the art, also includes meaning, as appropriate given the context of the usage, for example without limitation, data points and/or input and/or iterative input from an understanding of and/or knowledge of molecular chemistry, epigenetics, hormonal regulation, immunological mediators for example interleukins, the microbiome in and around the PFU, skin, the human body as related to the interactions of drugs and/or supplements and/or dietary supplements and/or nutrients and/or herbs and/or herbal treatments and/or dietary minerals and/or vitamins, and including an understanding of and/or knowledge of microbiome that is the bacteria of the human skin such as for example from the phylum of Gram-positive bacteria known as Actinobacteria such as corynebacteria and propionibacteria and micrococcia, from the phylum of mostly Gram-positive bacteria known as Firmicutes such as Staphylococca, from the phyla known as Bacteroidetes, Cyanobacteria, Proteobacteria, which populations of bacteria are found on various regions of the body, as recognized and understood by one of ordinary skill in the art.

The terminology used herein for describing particular illustrative example embodiments, is not intended to be limiting of the invention. Thus, as used herein, the singular forms “a” and “an” and “the” are intended to include the plural forms as well, unless the context clearly indicates otherwise. The terms “comprises” and “includes” and “comprising” and “including” when used in this specification, specify the presence of stated features, integers, steps, operations, elements, and/or components, but do not preclude the presence and/or addition of one or more other features, integers, steps, operations, elements, components, and/or groups thereof. The terms “description”, “describing”, “disclosure” and “disclosing”, as recognized and understood by one of ordinary skill in the art, also includes meaning the particular and detailed information provided herein regarding the invention as disclosed in the title, specification, claims, abstract, drawings, as readily recognized and understood by one of ordinary skill in the art, and these terms may be interchangeably as readily recognized and understood by one of ordinary skill in the art as appropriate given the context of the usage. The term “or” is intended to mean an inclusive “or” rather than an exclusive “or”, that is, unless specified otherwise, or clear from context, so that “X employs A or B” is intended to mean any of the natural inclusive permutations, namely, if X employs A; X employs B; or X employs both A and B, then “X employs A or B” is satisfied under any of the foregoing instances. Unless otherwise defined, all terms used herein, including technical and scientific terms, have the same meaning as recognized and understood by one of ordinary skill in the art in the art to which this disclosure belongs. Terms, such as those defined in commonly used dictionaries, should be interpreted as having a meaning that is consistent with their meaning in the context of the relevant art and should not be interpreted in an idealized and/or overly-formal sense unless expressly so defined herein. Terms may have meaning consistent with their meaning in the context of the relevant art to which this disclosure pertains as well as additional and clarifying meaning and nuance given to them as defined herein. Note that although terms “first” and “second” and similar ordinal numbers can be used herein to describe various steps and/or parts of methods and/or systems, these terms are not intended necessarily be limiting, rather these terms are used to distinguish certain steps and/or parts of methods and/or systems from certain others, and therefore, a first step, part or section discussed below could be termed a second step, part or section without departing from various teachings of this disclosure. Relative terms that are herein to describe the invention can be used to describe relationships such as disclosed herein, and are intended to encompass not only the relationships described herein and/or as depicted in the accompanying illustrative exemplary drawings, but also different relationships evident to and recognized and understood by one of ordinary skill in the art upon reading this disclosure. Terms listed below, as recognized and understood by one of ordinary skill in the art, are further defined for additional clarity.

The term “inflammatory skin condition”, as recognized and understood by one of ordinary skill in the art, also includes meaning, Acne vulgaris, acne, mask acne, acne inflammation, immunoinflammatory disorders, Pityriasis lichenoides chronica, Pityriasis rosea, alopecia areata, vitiligo, erythema multiforme, lichen planus, Hidradenitis suppurativa, atopic dermatitis, contact dermatitis, folliculitis, psoriasis, eczema, seborrhea, seborrheic dermatitis, dermatitis herpetiformis, and other inflammatory skin conditions, dermatological diseases, and also disorders wherein healthy tissue is compromised by an inflammatory process such as for example inflammation including for example arthritis and heart disease, and/or improper regulation of the immune system and/or autoimmune disorders such as Lupus and scleroderma, and/or undesired proliferation of cells, and includes other inflammatory conditions wherein there is a reduction and/or elimination of overall inflammation in the body of the patient for at least the reasons that the underlying causal factors of acne and other inflammatory skin conditions are addressed and/or reduction of any specific inflammation directed against a particular tissue and/or organ such as for example the skin and hair and related dermatological structures as described herein. The term “mask acne” that is “maskne”, as recognized and understood by one of ordinary skill in the art, also includes meaning, as appropriate given the context of the usage, a term that includes multiple skin conditions that appear to be associated with the wearing of a mask, which mask-wearing was part of the daily routine worldwide starting in early 2020 during beginning of the coronavirus pandemic, including for example without limitation, acne, rosacea, contact dermatitis, folliculitis, inflammatory skin conditions, and conditions wherein for example without limitation pores in the skin are clogged with oil, dead skin cells, dirt, and/or there is an allergic and/or sensitivity to a material of the mask.

The term “gut”, as recognized and understood by one of ordinary skill in the art, also includes meaning, as appropriate given the context of the usage, the digestive tract and/or intestinal tract and/or alimentary canal especially between the pyloric valve and the anus and or the small intestines, the large intestines, and the colon. The term “leaky gut”, as recognized and understood by one of ordinary skill in the art, also includes meaning, a lack of integrity with regard to the gut and/or intestinal lining and/or intestinal tract for example, without limitation, by opening the bonds between the cells of the intestinal barrier layer, such that the reduced integrity permits leakage from and/or translocation across the barrier of the lumen gut of products such as, but not limited to, food antigens, such as but not limited to colonic microbes and their byproducts, into, for example but not limited to, the area outside the lumen of the gut and/or the surrounding lymphatic system and/or into the bloodstream, which leakage may be causative of, for example but not limited to, inflammation, and especially inflammatory skin conditions such as acne as well as systemic inflammation and/or metabolic diseases and/or neurological diseases, also includes meaning, as per the context of usage, leakage from translocation across the barrier of the lumen, rather than by absorption, metabolism, and the transport of small molecules through the microvilli atop the lining cells of the intestinal barrier.

The term “yeast”, as recognized and understood by one of ordinary skill in the art, also includes meaning in the disclosure herein, the Kingdom Fungi and including fungi, mold and mushrooms, and may be used interchangeably, as per the context of usage, with the terms Candida, and/or Candida yeast and/or Candida albicans and/or Candida yeast and/or Malassezia yeast that is a cross-reactive yeast commonly present in and/or on the skin and/or skin-related tissues such as a hair follicle, and also includes meaning yeast and/or yeast byproducts such as for example as found in breads, cheeses, wines beers, nutritional yeasts, and yeast extracts. The term “microbiome”, as recognized and understood by one of ordinary skill in the art, also includes meaning, the microbes of an environment such as the gut including for example but not limited to bacteria, archaea, protists, viruses, yeast, fungi.

The terms “integrative”, “holistic”, “whole-system”, as recognized and understood by one of ordinary skill in the art, also include meaning, discerning causal factors of acne, of acne inflammation and of other inflammatory skin conditions, from multiple perspectives, for example but not limited to, dermatologic and immunologic and other different healing systems as described herein, and/or by treating using the safest and most effective methods before proceeding to more reaction-inhibitory or invasive methods with potentially more severe and likely side effects, and/or by including in the treatment patient self-care and understanding, and/or by including input from conventional medicine, as well as other forms of medicine, for example but not limited to, herbal, Ayurvedic, Chinese, homeopathic, functional, alternative, complementary, naturopathic, restorative, lifestyle, and other healing systems, and/or verifying likelihood, mechanism, and relevance in the scientific and medical literature to discern possible applicability of these inputs to patient treatment, and/or viewing acne and other inflammatory skin conditions as having a broad number of causal factors and viewing them as a collection of disorders and/or conditions, for example but not limited to, acne and other inflammatory skin conditions, that share a group of environmental and individual-specific etiologic factors. The terms “dietary supplements”, “nutrients”, “herbs”, “herbal treatments”, as recognized and understood by one of ordinary skill in the art, also include meaning those specifically disclosed herein in some embodiments of the invention.

The terms “causal factor” and “causal factors” of acne and other inflammatory skin conditions, as recognized and understood by one of ordinary skill in the art, also include meaning, “underlying causal factors of acne and other inflammatory skin conditions”, as disclosed and described in detail herein, and also include meaning the perspective and approach to acne and other inflammatory skin conditions and inflammation in the body, wherein they are seen not just as diseases, but as signs of underlying imbalance that leads to that is destined to lead to acne, other inflammatory skin conditions, and/or inflammation in the body. The term “sugar”, as recognized and understood by one of ordinary skill in the art, also includes meaning, as appropriate given the context of the usage, high-glycemic foods and/or simple starches as described and disclosed herein.

The term “PSU” means Pilosebaceous units, and as recognized and understood by one of ordinary skill in the art, also include meaning, comprising the hair follicle and/or hair bulb and associated sebaceous gland(s) and arrector pili muscle and, in appropriate contexts herein, associated apocrine gland(s) opening into the hair follicle and/or sweat glands, and as recognized and understood by one of ordinary skill in the art, also include meaning, sebaceous glands namely the multi-lobular holocrine glands found in all regions of the skin, except for the palmar and plantar surface, and as recognized and understood by one of ordinary skill in the art, also include meaning, sebaceous follicle and follicular unit, as well as the resident microbiome in that area of the Pilosebaceous units.

The terms “patient data points” and “data points” are used in this disclosure to mean data points that relate to and/or are relevant to and/or are pertinent to and/or the treatment of the patient, for example but not limited to, data points collected and/or organized and/or interpreted from patient queries such as during initial and subsequent meetings and/or examinations, patient history such as prior medical treatments and/or family history, medical testing, as recognized and understood by one of ordinary skill in the relevant medical art, for example, diagnostic tests, blood tests, bodily fluids sampling and analysis, imaging, body function measurements, biopsy, bacterial culture, analysis of genetic material, Applied Kinesiology, methane-hydrogen breath testing, essential minerals testing, stool microbiome testing, conventional medicine testing, traditional medicine testing, functional medicine tests, food allergy and/or food sensitivity testing whether in vitro or in situ, differential complex sugar absorption testing, examination of tongue and/or nails, examination of frenulum, postural analysis, analysis of emotional and/or psychological and/or physical characteristics, observation of patient, examination of patient, evaluation of patient, knowledge of casual factors of acne and/or acne inflammation and/or other inflammatory skin conditions as described herein, knowledge outside of science and yet corroborated by science, knowledge of real-time data relating to the patient after initial queries are conducted, knowledge of medicines such as conventional medicine and/or traditional medicine including skills and practices of various cultures and/or complementary medicine including traditional Chinese medicine and Ayurveda medicine and homeopathy and naturopathy and mind/body practices and natural health products including herbs and dietary supplements and probiotics and prebiotics and/or alternative medicine and electrodiagnostic tools, functional medicine, restorative medicine, precision medicine, and knowledge of other healing systems.

The multiple embodiments of the invention, that is, the methods and systems for treatment of acne and other inflammatory skin conditions as described herein in the specification, drawings, examples and claims, disclose novel applications of relationships between multiple data points collected, organized, interpreted, processed, structured and/or related, which multiple data points relate to a patient in need of treatment for acne and other inflammatory skin conditions. In embodiments of the invention, including those of the illustrative examples below, the novel application of relationships between multiple patient data points lead to safe, effective, integrative, holistic, whole-system methods and systems for treating acne and other inflammatory skin conditions that provide, inter alia, multiple advantages readily recognized and understood by one of ordinary skill in the art based upon this disclosure, and provides solutions to the multiple disadvantages and problems of known methods as described above, and which novel applications of relationships between multiple patient data points safely and effectively addressing the issues underlying the symptoms observed in a integrative, holistic, whole-system way. As a result of utilizing the novel methods and systems disclosed herein, it was surprisingly discovered, as described in the illustrative examples herein, for example but not limited to, an unexpected resultant synergy and/or restoration of balance in the patient's body so as to provide integrative, useful, safe and effective treatment of acne and other inflammatory skin conditions.

In an embodiment of the invention, multiple patient data points were collected, organized, interpreted, processed, structured and/or related and relationships were determined between multiple patient data points and relationships between multiple patient data points were applied, including for example, by formulating treatment options and adjusting treatment options in view of the relationships between multiple patient data points and structuring a treatment protocol from the treatment options and administering the treatment protocol to the patient and monitoring the patient for response to the administered treatment protocol and optionally reiterating one or more steps of the invention, thereby solving multiple disadvantages and problems of known methods as described above.

In an embodiment of the invention, multiple patient data points were collected, organized, interpreted, processed, structured and/or related and relationships were determined between multiple patient data points and relationships between multiple patient data points were applied, including for example, by formulating treatment options and adjusting treatment options in view of the relationships between multiple patient data points and structuring a treatment protocol from the treatment options and administering the treatment protocol to the patient and monitoring the patient for response to the administered treatment protocol and then optionally iterating selection of the steps of collecting, organizing, interpreting, processing, structuring, and/or relating multiple patient data points, and/or the step of determining relationships between multiple patient data points, and/or the step and applying relationships between multiple patient data points, including for example, by formulating treatment options and adjusting treatment options in view of the relationships between multiple patient data points, and/or the step of structuring a treatment protocol from the treatment options, and/or the step of administering the treatment protocol to the patient, and/or the step of monitoring the patient for response to the administered treatment protocol wherein the step of iterating one or more of the steps results in having the effect of, for example but not limited to, enhancing structuring of an integrative, useful, safe and effective treatment protocol structured to address the underlying issues and underlying causal factors of acne and/or acne inflammation and/or other inflammatory skin conditions, and/or in the case of repeating one or more of the step collecting, organizing, interpreting, processing, structuring, and/or relating multiple patient data points, and/or the step of determining relationships between multiple patient data points, having the effect of revealing the temporal relationships between multiple data points leading for example but not limited to determining case and effect relationships and/or having the effect of revealing patterns in the multiple data points for example but not limited to multiple incidents of increased acne breakouts following ingestion of foods or other items that are one or more of the causal factors of acne and other inflammatory skin conditions, thereby resulting in successfully reducing and/or eliminating the patient's acne and/or acne inflammation and/or other inflammatory skin condition, thereby solving multiple disadvantages and problems of known methods as described above.

In yet another embodiment of the invention, multiple patient data points were collected, organized, interpreted, processed, structured and/or related and relationships were determined between multiple patient data points and relationships between multiple patient data points were applied, including for example, by formulating treatment options and adjusting treatment options in view of the relationships between multiple patient data points and structuring a treatment protocol from the treatment options and administering the treatment protocol to the patient and monitoring the patient for response to the administered treatment protocol and then optionally iterating selected of the steps of collecting, organizing, interpreting, processing, structuring, relating, determining, applying, formulating, adjusting, structuring, monitoring, wherein the structuring of the treatment protocol resulted in creating an integrative, useful, safe and effective treatment information regarding a patient organized to develop an integrative, useful, safe and effective treatment protocol structured to address the underlying causal factors of acne and other inflammatory skin conditions thereby resulting in successfully reducing and/or eliminating the patient's acne or other inflammatory skin condition, and especially but not limited to, by fixing the patient gut dysfunction, thereby solving multiple disadvantages and problems of known methods as described above.

In yet another embodiment of the invention, in particular regarding the step of determining and/or applying relationships between multiple patient data points, included for example but not limited to reducing and/or eliminating the overgrowth of Candida yeasts in the gut of the patient, such as Candida albicans in recognition that, for example but not limited to, yeast food products ingested by the patient result in yeast overgrowth in the gut leading to improper permeability of the intestinal lining, that is, leaky gut, and leading to food antigens leaking out of the gut into the bloodstream causing inflammation both in and around the gut liming as well as elsewhere in the body including at the skin as described herein, thereby solving multiple disadvantages and problems of known methods as described above.

In yet another embodiment of the invention, in particular regarding the step of determining and/or applying relationships between multiple patient data points, included for example but not limited to queries regarding the patient's antibiotic usage that may indicate a relationship between one or more causal factors of acne and other inflammatory skin conditions and an alteration of the normal bacteria in the gut that permits the overgrowth of yeast and related inflammation, wherein the gathering of additional patient data would relate to fiber in the patient diet that would relate to another relationship in that fiber provides a matrix on which more diverse bacteria can grow thus reducing the likelihood of yeast overgrowth, including fiber-containing food products such as vegetables and/or bran bind and carry yeast breakdown products and/or bile with lipid-soluble excretory products out of the digestive tract, and also another relationship in that when the intestinal lining permits leakage of food allergens into surrounding blood supply for the small intestines and lymphatic tissues, that is, the Peyer's patches, these allergen food fragments then have an impact on one of the largest collections of lymphatics cells in the body's immune system, thereby solving multiple disadvantages and problems of known methods as described above.

In yet another embodiment of the invention, in particular regarding the step of determining relationships between multiple patient data points, such relationships determined may include, for example but not limited to recognizing that microbes of the gut are influenced by and/or respond to the environment in which the patient lives and not just by genetics, and that microbial and/or bacterial metabolites, for example, nicotinamide that is a form of vitamin B-3, are related to metabolic pathways that produce energy in cells, and that microbial and/or bacterial metabolites may be utilized by other microbes in the gut, and that there are positive health benefits of balancing microbial species in the gut, and that bacteria often work in communities in that they are social organisms with interactions between and among species and may occupy a common physical location, and that the gut microbiome may include microbes such as bacteria, archaea, protists, viruses and fungi that are unique and/or different for each patient and yet “normal” for each such patient, thereby solving multiple disadvantages and problems of known methods as described above.

In yet another embodiment of the invention, in particular regarding the step of determining and applying relationships between multiple patient data points, included for example but not limited to, queries to obtain additional multiple patient data points that relate to possible incomplete digestion and reveal another relationship, in that incomplete digestion results in larger fragments of incompletely digested proteins of food leaking out of the gut into the immune system lymphatic tissues and/or blood supply for the small intestines and, as some of these larger fragments have patterns of amino acids that are recognizable by the immune system, the immune system may react against structures with similar patterns in the body thereby resulting in inflammation and autoimmune reactions and including acne and other inflammatory skin conditions. Application of these relationships includes using demulcents to coat the lining of the intestinal tract, for example, products that contain protective substance that coats the digestive tract, such as deglycyrrhizinated licorice, okra, fenugreek seed tea, marshmallow root tea, and aloe, and also avoiding use of medications that inhibit production of secretion of protective mucus layer that protects the inner surface of the intestines, such as non-steroidal anti-inflammatory drugs like ibuprofen, aspirin, and protein pump inhibitors like esomeprazole. An application of these relationships also includes maintaining and supporting liver function to provide a cleansing route for any food allergens in the bloodstream that might result in inflammation, and also reducing the growth of yeast by using anti-yeast supplements and/or clearing mold from the home environment. Yet another application of these relationships include creating treatment information that incorporates recognizing food antigens can be molecular mimics of antigens found in the skin and can stimulate the lymphoid tissue around the intestines to react against not only such lymphoid tissue, but also the immunologically-analogous structures in the peri-follicular and/or sebaceous gland apparatus, causing acne and other inflammatory skin conditions, and including for example determining foods that are immunologically related to specific pollen to which the patient has a known sensitivity. After multiple queries to collect patient data, such relationships were determined and applied in novel ways to create treatment information then organized to develop a treatment protocol then structured to address the underlying causal factors to treat acne and other inflammatory skin conditions, thereby solving multiple disadvantages and problems of known methods as described above.

In yet another embodiment of the invention wherein a personalized method of treatment for a patient with acne or other inflammatory skin condition is provided, comprising the steps of collecting multiple patient data points by queries of the patient and by measuring relevant physical information as indicated by the queries and determining whether one or more causal factors for acne or other inflammatory skin conditions as indicated based on the collected multiple patient data points and determining relationships between the multiple patient data points and applying these relationships to create treatment information regarding the patient and organizing the treatment information and developing a treatment protocol structured to address the underlying causal factors and administering such treatment protocol and thereby resulted in successfully reducing and/or eliminating the patient's acne or other inflammatory skin condition, thereby solving multiple disadvantages and problems of known methods as described above.

In yet another embodiment of the invention, the relationships between multiple patient data points included adjusting the treatment protocol to match the individual patient based on the patient data points whether gathered from patient queries and/or patient medical test results. Thus, the patient treatment was a specific treatment protocol anchored in the specific and comprehensive multiple patient data points gathered, processed, interpreted, organized, structured and related that led to the determination of relationships that were then applied to create the treatment information that formed the treatment protocol. The multiple patient data points gathered, processed, interpreted, organized, structured, related in reference to and/or in view of, causal factors of acne and/or acne inflammation as described herein, and in addition to those known in the art. Thus, the patient treatment protocol was adjusted by iteration of one or more steps of the invention wherein by for example but not limited to the patient experience and/or response to the administered treatment protocol was collected as an additional one or more data points that again were organized, interpreted, processed, structured and/or related and relationships were determined between multiple patient data points such as but not limited to reinforcing the relationships already determined between other multiple data points, and/or correlating the relationships already determined between other multiple data points, and include for example but not limited to exacerbation of patient acne and/or other inflammatory skin condition after ingesting foods and/or other items that are causal factors of acne and/or other inflammatory skin conditions such as milk, cheese, fried foods, sugars as defined herein including high-glycemic foods and simple starches, high-temperature heated fats, coffee, stimulants, wheat gluten, junk food diet foods.

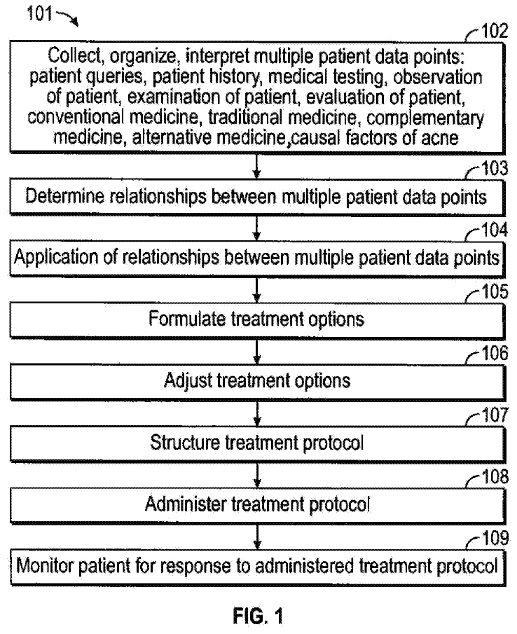

FIG. 1 shows, in schematic form of a flowchart, an illustrative, exemplary embodiment 101 of the invention wherein the steps comprise collecting, organizing, interpreting multiple data points 102 using various inputs, such as patient queries, patient history, medical testing, observation of patient, examination of patient, evaluation of patient, conventional medicine, traditional medicine, complementary medicine, alternative medicine, and causal factors of acne, and determining 103 relationships between multiple data points, and applying 104 relationships between multiple data points by formulating 105 treatment options and adjusting 106 treatment options in order to structure 107 a treatment protocol and administer 108 treatment protocol to the patient, and monitor 109 patient for response to administered treatment protocol.

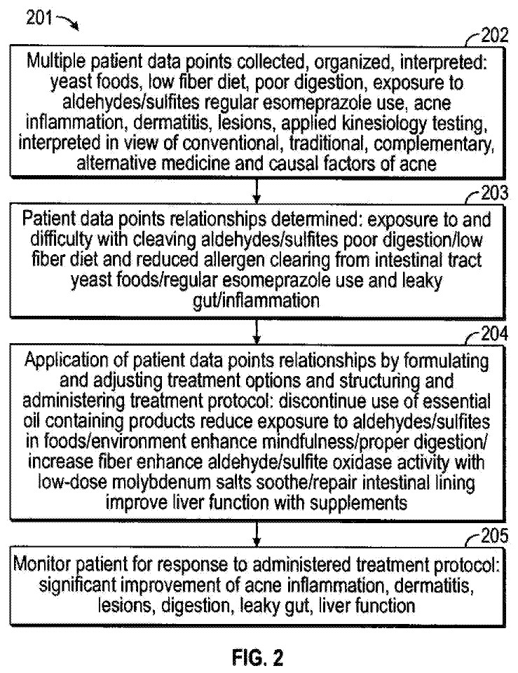

FIG. 2 shows, in schematic form of a flowchart, an illustrative, exemplary embodiment 201 of the invention wherein the steps comprise collecting, organizing, interpreting multiple data points 202 using various inputs that include the patient's ingestion of yeast foods, patient's low fiber diet and poor digestion, patient's exposure to aldehydes and/or sulfites, patient's regular u of esomeprazole, patient's acne inflammation and dermatitis and lesions, and testing using Applied Kinesiology and data points interpreted in view of conventional medicine, traditional medicine, complementary medicine, alternative medicine and causal factors of acne, and determining 203 relationships between multiple data points, including patient's exposure to and difficulty with cleaving aldehydes and/or sulfites, poor digestion and/low fiber diet and reduced allergen clearing from intestinal tract, and yeast foods consumption and regular esomeprazole use and leaky gut and inflammation and applying 204 relationships between multiple data points by formulating 205 treatment options and adjusting 206 treatment options in order to structure 207 a treatment protocol, including discontinued use by patient of essential oil containing products, reducing patient exposure to aldehydes and/or sulfites in foods and/or environment, enhancing patient mindfulness and proper digestion and increased fiber in diet, enhancing aldehyde and/or sulfite oxidase activity of patient with low-dose molybdenum salts, soothing and repair intestinal lining of patient, improving patient liver function with supplements, and administering 208 treatment protocol to the patient and monitoring 209 patient for response to administered treatment protocol wherein, for example but not limited to, there was improvement of patient acne inflammation, dermatitis, lesions, digestion, leaky gut, liver function.

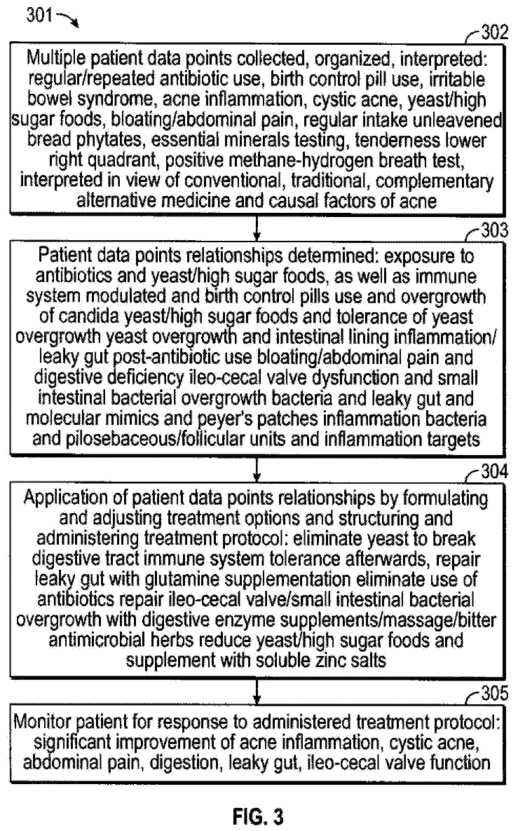

FIG. 3 shows, in schematic form of a flowchart, an illustrative, exemplary embodiment 301 of the invention wherein the steps comprise collecting, organizing, interpreting multiple data points 302 using various inputs, such as patient's regular and repeated antibiotic use, patient use of birth control pills, patient's irritable bowel syndrome, patient's acne inflammation and cystic acne, patient's ingestion of yeast foods and high sugar foods, patient's bloating and abdominal pain and regular intake of unleavened bread phytates, essential minerals testing, patient tenderness in the lower right quadrant, patient positive methane-hydrogen breath test, interpreted in view of conventional medicine, traditional medicine, complementary medicine, alternative medicine and causal factors of acne, and determining 303 relationships between multiple data points, including patient exposure to antibiotics and yeast-containing and high sugar foods, as well as the patient's immune system modulation and birth control pills use and overgrowth of Candida, and consumption of yeast-containing and/or yeast-byproduct-containing and/or high sugar foods and/or high glycemic foods in relation to tolerance of yeast overgrowth and yeast overgrowth in relation to intestinal lining inflammation and/or leaky gut and post-antibiotic use in relation to patient bloating and abdominal pain and patient digestive deficiency and patient ileocecal valve dysfunction in relation to small intestinal bacterial overgrowth and patient's intestinal bacteria and/or colonic bacteria and ileo-cecal valve dysfunction and leaky gut in relation to molecular mimics in patient's body and in relation to Peyer's patches inflammation and patient's intestinal bacteria and/or colonic bacteria and ileo-cecal valve dysfunction and leaky gut in relation to pilosebaceous and/or follicular units and inflammation targets, and which both disperses via the blood throughout the body and acts as a target and/or sensitizes the immune cells in the Peyer's patches around the small intestines, and applying 304 relationships between multiple data points by formulating and adjusting treatment options and structuring and administering treatment protocol, including eliminating yeast from patient's diet to break digestive tract immune system tolerance, as well as stopping yeast-induced inflammation of the small intestines, and afterwards repairing leaky gut with glutamine supplementation and eliminating use of antibiotics and repairing patient ileo-cecal valve and small intestinal bacterial overgrowth with digestive enzyme supplements and lower right quadrant massage and bitter antimicrobial herbs and reducing patient's intake of yeast-containing and/or high sugar foods and supplement diet with soluble zinc salts and monitoring patient 305 for response to administered treatment protocol wherein there was significant improvement of patient's acne inflammation and cystic acne, abdominal pain, digestion, leaky gut, and ileo-cecal valve function.

Note that some embodiments of the methods and systems of the invention include iterations of one or more of the steps of the invention including for example but not limited to iterative queries of the patient and/or iterative input from different medicines and/or healing systems as described herein such as conventional medicine, traditional medicine, complementary medicine, alternative medicine, functional medicine, restorative medicine, precision medicine, and/or reconsideration of the causal factors of acne and/or acne inflammation, and therefore, such iterations of one or more steps can take the form of a computer program product accessible by a computer-usable or computer-readable medium or memory providing program code for use by or in connection with a computer program product or any instruction execution system as recognized and understood by one of ordinary skill in the art. Thus, some embodiments of the invention may utilize artificial intelligence and/or machine learning and/or augmented intelligence and/or intelligence augmentation, as recognized and understood by one of ordinary skill in the art, in that these systems are utilized to conduct iterations of one or more steps of the invention and/or iterative inputs of data points as described herein, thereby solving multiple disadvantages and problems of known methods

Some embodiments of the methods and systems of the invention incorporate in one or more of the steps some amount of artificial intelligence and/or machine learning and/or augmented intelligence and/or intelligence augmentation by utilizing information technology, as recognized and understood by one of ordinary skill in the art, for example but limited to, augmenting analysis and/or decisions of the person and/or persons using the methods and systems of the invention, assisting analysis and/or decisions of the person and/or persons using the methods and systems of the invention, enhancing analysis and/or decisions of the person and/or persons using the methods and systems of the invention, reinforcing analysis and/or decisions of the person and/or persons using the methods and systems of the invention, guiding analysis and/or decisions of the person and/or persons using the methods and systems of the invention, strengthening analysis and/or decisions of the person and/or persons using the methods and systems of the invention, corroborating analysis and/or decisions of the person and/or persons using the methods and systems of the invention, simplifying analysis and/or decisions of the person and/or persons using the methods and systems of the invention, the steps of the methods and systems of the invention being those described herein, namely, for example but not limited to, one or more of the steps of collecting, organizing, interpreting, processing, structuring, relating, determining, applying, formulating, adjusting, structuring, administering, monitoring. Some embodiments of the methods and systems of the invention incorporating in some and/or all of the steps artificial intelligence and/or machine learning and/or augmented intelligence and/or intelligence augmentation by utilizing information technology, as recognized and understood by one of ordinary skill in the art, utilize such information technology, that is artificial intelligence and/or machine learning and/or augmented intelligence and/or intelligence augmentation, by addressing large data sets, for example but not limited to, large data sets of multiple patient data points wherein relationships between multiple patient data points are detected and/or determined, and wherein such large data sets of multiple patient data points necessitate non-traditional data processing, that is artificial intelligence and/or machine learning and/or augmented intelligence and/or intelligence augmentation, and wherein artificial intelligence may be employed to mimic the behavior of the person and/or persons using the methods and systems of the invention, and wherein machine learning may be employed to utilize statistical techniques that assist the information technology learn by experience the methods and systems of the invention, including, for example but not limited to, one or more of the steps of collecting, organizing, interpreting, processing, structuring, relating, determining, applying, formulating, adjusting, structuring, administering, monitoring, thereby solving multiple disadvantages and problems of known methods as described above.

In an embodiment of the methods and systems of the invention, an algorithm constructed from one or more steps of an embodiment of the invention including but not limited to, as described herein, the steps of collecting, organizing, interpreting, processing, structuring, relating, determining, applying, formulating, adjusting, structuring, administering, monitoring, and included iterations of one or more of the steps of the embodiment of the invention, wherein for example but not limited to, wherein the step of collecting was repeated to collect additional multiple data points and/or wherein the step of organizing was repeated to organize multiple data points and/or wherein the step of interpreting was repeated to interpret multiple data points and/or wherein the step of processing was repeated to process multiple data points and/or wherein the step of structuring was repeated to structure multiple data points and/or wherein the step of relating was repeated to relate multiple data points and/or wherein the step of determining was repeated to determine relationships between multiple data points and/or wherein the step of applying was repeated to apply determined relationships and/or wherein the step of formulating and the step of adjusting were repeated to formulate and apply treatment options and/or wherein the step of structuring was repeated to structure a treatment protocol and/or wherein the step of administering was repeated to administer a treatment protocol and/or wherein the steps of monitoring was repeated to monitor response of patient to treatment protocol, and wherein the one or more steps iterated were repeated one or more times and singly or in groups of one or more steps, and which for example but limited to, inter alia, superimposed multiple data points relating to principles from multiple healing systems and thereby added perspective to treatment of acne and other inflammatory skin conditions by utilizing a multi-dimensional database of multiple data points collected, organized, interpreted, processed, structured, related, and thereby solving multiple disadvantages and problems of known methods as described above, including but not limited to approaching treatment from a limited and/or non-integrated and/or piecemeal perspective and/or delaying evaluation and correction of for example causal factors of acne and other inflammatory skin conditions which delays often lead to other health problems, that is, co-morbidities, and negative health issues.

In another embodiment of the methods and systems of the invention, an algorithm constructed from selected steps of the invention including but not limited to, as described herein, the steps of collecting, organizing, interpreting, processing, structuring, relating, determining, applying, formulating, adjusting, structuring, which included repeating patient queries relating specifically to relevant aspects of the causal factors of acne and/or acne inflammation and/or other inflammatory skin conditions, and repeating the steps of collecting and/or organizing and/or interpreting and/or processing and/or structuring and/or relating and/or multiple patient data points, and repeating the step of determining additional relationships between multiple patient data points, and repeating the step of applying these additional relationships by, for example, formulating and/or adjusting treatment options in relation to the relationships between multiple patient data points, and repeating the step of structuring a treatment protocol. In the embodiment, the algorithm resulted in at least one or more of the following: further querying, augmenting, enhancing, reinforcing, guiding, strengthening, the selected steps of the embodiment of the invention wherein the patient was determined to have antigens and/or allergens such as incompletely digested food and/or bacteria relating to ileo-cecal valve dysfunction wherein colonic material and bacteria would back up into the small intestines, which antigens and/or allergens leaked through patient's permeable intestinal lining, enter the bloodstream and subsequently be delivered to the PSU where they acted as targets and/or as stimulants for an inflammatory response as described herein, and wherein the patient was determined to have exposure to aggravators such as infections, foreign bodies, pseudo-hormones, and certain chemicals like topical cutting oils known to aggravate acne and/or acne inflammation when applied topically, and wherein the patient was determined to have exposure to environmental toxins that suppressed host-inappropriate inflammatory responses, and wherein there were cross-reactive responses to molecular mimics that stimulated immune responses in the patient that incited inflammatory recognition by a component of the immune system namely desquamating cells from sebaceous gland secretions as a result of their similarity to cells from milk-producing glands, thereby solving multiple disadvantages and problems of known methods.

In yet another embodiment of the methods and systems of the invention, an algorithm constructed from selected steps of an embodiment of the invention, which included repeating patient queries relating to relevant aspects of the causal factors of acne and/or acne inflammation and/or other inflammatory skin conditions, and repeating the steps of collecting, organizing, interpreting, processing, structuring, relating, multiple patient data points, and repeating the step of determining additional relationships between multiple patient data points, and the step of applying these additional relationships by and repeating the step of structuring a treatment protocol. In the embodiment, the algorithm resulted in augmenting, enhancing, reinforcing, guiding, strengthening the selected steps of the embodiment of the invention wherein the patient with acne and/or acne inflammation completed an initial general questionnaire regarding patient history and lifestyle and then completed a specific questionnaire regarding the same and created a timeline regarding the onset and changes in their skin condition and what preceded those events. Repeated analysis of these, as well as other, multiple patient data points, led to determining relationships between the multiple patient data points collected during repeated patient queries, including for example, the patient's diet and environmental toxin exposures and immune system function. The iterative queries were conducted to gather more patient data utilized to corroborate and/or to refute hypotheses of causal sequence. Repetition of selected steps of an embodiment of the invention resulted in additional determinations of relationships between multiple patient data points and implicated a particular causal pattern, for example but not limited to, patient yeast infections and worsening rash after antibiotic and birth control pills and a diet high in sugar and yeast, which additional multiple relationships are used to formulate a treatment program that addresses the causes underlying the health issues, for example, treatment including a diet low in sugar and simple carbohydrates that stops feeding the yeast growth, and also a diet eliminating foods containing yeast and/or yeast byproducts that tolerize the gut immune system to yeast, and also following such diet changes by supporting liver function and increasing fiber, as well as adding anti-yeast supplements and probiotics to patient's diet helping to repopulate the gut with a more balanced microbiome and a normal flora for the patient. In addition, a patient history of repeated and long-term antibiotic usage led to queries resulting in multiple patient data points such as yeast infections after the antibiotics, worsening of the condition after antibiotics, that indicates increased yeast in the microbiome, and worsening of the condition after taking steroids and birth control pills as well as patient data points regarding gastrointestinal symptoms such as bloating, gas, abdominal pain, constipation or diarrhea following use of steroids and birth control pills. Additional queries include whether celebrations or holidays or birthdays preceded aggravation of the gastrointestinal symptoms or acne or other inflammatory skin condition, and whether such aggravation involved increased sugar, that would feed yeast, or wine, beer, bread, or cheese, that would tolerize the gut immune system and favor yeast overgrowth. Further queries include what foods might have been also eaten that might be known to or suspected to cause aggravation, and see if there are similarities between those foods and know food allergens from the patient based on previous patient data gathered or lab testing or family allergy history. The resultant data gathered from the iterative queries and the additional relationships recognized and determined, all lead to an expanding hypothesis of “how it works” for the patient in that the causal factors of the condition for this patient are understood, and such findings point to specific causal factors, such as conditions increasing the chances of yeast overgrowth, like repeated or prolonged antibiotics, which then determine additional queries about other related causal factors and their temporal relationship to intestinal disturbances and onset of acne and/or other inflammatory skin conditions, thereby solving multiple disadvantages and problems of known methods as described above.

In yet another embodiment of the invention, in particular regarding the aspects of the step of multiple patient data points collected, organized, interpreted, processed, structured and/or related and the step of relationships determined between multiple patient data points, namely, multiple determined relationships pointed to the same etiology and therefore increased the probability of an underlying etiology being valid, and multiple queries of the patient history revealed repeated sequential reactions in the form of for example but not limited to acne breakouts following ingestion of species of foods, and multiple queries also refreshed patient memory regarding similar reactions to other substances, chemicals, and/or products, and Applied Kinesiology testing found weakening of a strong muscle when a food is placed under their tongue, or under a magnet over their solar plexus, wherein subsequent examination of the ingredients on the label of such food revealed ingredients known as one or more causal factors of acne and/or other inflammatory skin conditions.

In yet another embodiment of the invention, in particular regarding the aspects of the step of multiple patient data points collected, organized, interpreted, processed, structured and/or related and the step of relationships determined between multiple patient data points and the step of applying the determined relationships between multiple data points by the step of formulating and adjusting as needed treatment options, namely, multiple data points were collected, organized, interpreted, processed, structured and/or related utilizing Applied Kinesiology, patient history, conventional medical testing, and in light of pattern analysis to refine application of the determined relationships between multiple data points to formulate and adjust treatment options, namely, by selecting the most effective and overall least reactive way forward, for example but not limited to supplementing patient's dysfunctional organs, where the patient's digestive issues presented as a history of bloating, gas, abdominal pain after eating, nausea, diarrhea, and/or constipation. The patient was determined to be sensitive to gluten and/or dairy products, and an enzyme chosen that has specific activity for breaking down gluten and/or casein. Thus, both patient history and Applied Kinesiology were utilized in relating multiple data points in order to determine relationships between multiple data points and to formulate treatment options and to adjust them. And, for example, the enzyme chosen for assisting in the breakdown of gluten protein contained high concentrations of protease dipeptidyl peptidase IV, which contains molecular remnants of the Aspergillus mold from which the enzyme was derived, and so based on determined relationships between the patient data points, the formulated treatment option was adjusted and the patient was deemed better treated with a pancreatic or fruit-derived enzyme such as bromelain. Also, pattern analysis revealed multiple sources of data points leading towards the conclusion that patient leaky gut was induced by for example gluten and Candida and were some of the underlying causal factors of patient's acne exacerbations. Aggravation by very different previous treatments such as antibiotics or birth control pills, or their withdrawal, were suggestive of causal factors that increase yeast overgrowth in the gut and irritate and enflame the gut mucosa leading to leaky gut. Patient's gastrointestinal symptoms during or after using antibiotics or birth control pills, or after having a high glycemic meal or diet, or after acute or chronic consumption of high yeast/mold containing or yeast/mold derived foods such as bread, cheese, wine and beer also supported the hypothesis that Candida overgrowth was a factor in causing patient leaky gut that led to the antigenic exposure aggravating the inflammatory attack on the PSU manifesting as acne. Thus, the invention provides safe, effective, integrative, holistic, whole-system methods and systems for treating acne and other inflammatory skin conditions that provides, inter alia, multiple advantages readily recognized and understood by one of ordinary skill in the art based upon this disclosure, and provides solutions to the multiple disadvantages and problems of known methods as described above, in one embodiment, by the iteration of the step of multiple patient data points being collected, organized, interpreted, processed, structured and/or related and the step of relationships being determined between multiple patient data points and the step of applying the determined relationships between multiple data points by the step of formulating and adjusting as needed treatment options.

In some embodiments of the invention, in particular regarding the aspects of the step of multiple patient data points and/or input and/or iterative input collected, organized, interpreted, processed, structured and/or related and the step of relationships determined between multiple patient data points and the step of applying the determined relationships between multiple data points by the step of formulating and adjusting as needed treatment options, namely, multiple data points are in part collected utilizing an apparatus that provides, for example and without intending to be limiting, data points and/or input and/or iterative input of the patient at the atomic and/or molecular level, the energetics and dynamics of enzymatic reactions, charge and topography structure of interactive binding sites such as by freeze fracture atomic microscopy and in silico, conformation changes with binding information, micro-energetics of biochemical reactions, and in such embodiments of the invention, the multiple patient data points and/or input and/or iterative input collected, organized, interpreted, processed, structured and/or related, as recognized and understood by one of ordinary skill in the art, also includes meaning, as appropriate given the context of the usage, for example without limitation, data points and/or input and/or iterative input from an understanding of and/or knowledge of the biochemical reactivity energetics at the electromagnetic level, an understanding of and/or knowledge of measurements of homeostasis and/or harmonic stability and/or dysfunction, an understanding of and/or knowledge of assessing disease or wellness by evaluating measurements of homeostasis and/or harmonic stability and/or dysfunction with specific dynamic challenges and/or with correlations and/or relationships across multiple assessment methodologies, for example with limitation, electrodermal testing, acupuncture pulse reading, readings using devices such as Asyra and/or Scenar, an understanding of and/or knowledge of prior art apparatuses and platforms for detection, an understanding of and/or knowledge of meditative and/or awareness and/or spiritual and/or Eastern energetic-based techniques, and in such embodiments of the invention the apparatus includes for example without limitation an information and electromagnetic spectrum measuring system that noninvasively detects conditions, diseases, illnesses including for example bacterial and/or viral infections such as for example coronavirus including the novel coronavirus known as severe, acute respiratory syndrome coronavirus 2 that is SARS-CoV-2 that is HCoV-19 and/or influenza, and the current viral, immune and enzymatic infectious stage, inflammation, inflammatory conditions, inflammatory skin disorders, acne, and other non-well states of the patient, as recognized and understood by one of ordinary skill in the art, based upon the disclosure herein, and in such embodiments of the invention the apparatus collects multiple static and post-challenge data points and/or input that is then assessed for example with probability tools, and for example to make stochastic evaluation, and is assessed with probability tools as it clusters wellness or disease-wise according to an overall treatment path, wherein the efficacy of predictive data for the individual and/or individuals with similar data clusters are assessed for benefit and compared and recalibrated with outcome, and wherein strong data protection techniques are utilized to prevent misuse and/or leaked data, including for example collecting data relating to environment, diet, infectious trends, socially-pressured trends, oppressive trends, then evaluated for financial and emotional cost, explored for remedies, and then remedied, and focusing on providing improved human life both quantitatively and qualitatively and less oppressive for everyone.

In such embodiments of the invention, the apparatus greatly increases accuracy and effectiveness and safety of selection of, when compared to shortcomings of devices in the prior art, multiple pharmacologic, nutritional and/or herbal supplements and combinations, for example by registering improvement of measured parameters both rapidly and non-invasively, wherein for example the measured parameters include electromagnetic and/or spectral emission characteristics of the patient's condition, including for example inflammatory skin conditions and/or inflammations and/or infections including bacterial and viral and including infection by the SARS-CoV-2 virus that are used to assess resonant patterns from various stages of disease and/or infection, and in such embodiments of the invention the apparatus measures and/or assesses and/or evaluates, and optionally together with a human operator, resonant patterns that is salient segments, that are emitted from the apparatus to determine whether they are affected at the molecular, cellular, animal and/or human level wherein multiple parameters of homeostasis are measured and/or assessed and/or evaluated, such as temperature, arterial blood gas including pH, PO2, PCO2, base excess and bicarbonate as recognized and understood by one of ordinary skill in the art, heartrate variability, glavinic skin response, resistance including capacitance across the body and/or at specific sites utilizing various frequency patterns, in response to electronic signatures of the treating modality coded into spectral frequency patterns such as radio waves, and then the salient patterns would be emitted from the apparatus to determine whether resonance diminished homeostasis of the measured patient, and wherein disruptive patterns are assessed and/or evaluated as well as effects of natural substances and drugs known or suspected to interfere with, for example, particular steps in an infective process, or particular stages of a disease state, and wherein based on these data points and/or input a probability is established as to whether the patient's condition was in disease state as described herein, for example, one or more inflammatory skin conditions and/or inflammation and/or infections including bacterial and viral and including infection by the SARS-CoV-2 virus, and if present, at what stage of disease state, and in such embodiments of the invention the apparatus is constructed as a receptor-reporter device using lectins and/or sugars that are bound, for example, with sialic acid bound on a graphene membrane, with a reporter molecule attached that changes conformation and/or changes charges upon binding that then triggers a signal both repeated and amplified for the detection signal, and/or the apparatus is constructed to measure and/or to assess and/or to evaluate energies and determine salient signatures and/or to measure and/or to assess and/or to evaluate data and/or input and/or information relating to the following, as described and disclosed herein and as recognized and understood by one of ordinary skill in the art based on the disclosure herein, including for example: structure of enzymes and/or molecular steps involved in infection process and/or subversion of host cell systems; in silico simulations of those steps and predicted activation energies and/or energies of reactions; atomic reactions by charge, by docking, and by energy when binding down including at for example nano levels, pico levels, femto levels and/or atto levels; isolated chemical reactions; cell cultures in the process of infection; cell cultures in the process of infection while inhibiting successive steps of infection to isolate specific steps using known inhibitors of those steps; summation effects and activation of conformational changes by binding that activate enzymatic or other processes; molecular interactions that are with or known to be or be activated or inhibited by light spectra; magnetic or other spectral reactions or their sequential patterns with frequency and magnitude data; enhancing patterns by electromagnetic or other frequency-mimicking energies such as for example by enhancing activation energy of specific reactions, and observing whether the viral process increases or the specific reaction, cell, animal, or patient shows signs of worsening instantaneously by galvinic skin response, heart rate variability, blood oxygen that is P02, and interfering with those patterns to see if signs of improvement occur; assessment of summations of individual spectral signatures of a multitude of cells undergoing the same process at similar but staggered times, and the progression to numerous steps in the sequence of multiple steps occurring in the progression, and looking for discernable patterns, and patterns of nodes of additive energies that are brighter and more readily detectable, as well as the shift of those nodes and nodal patterns with calculated input to the system, and recognizing those patterns and comparing them with similar patterns from for example without limitation, other viruses and conditions to help distinguish a particular virus or condition; known drugs, herbs, chemical, and/or biological substances or pH changes to inhibit various steps in the reaction to isolate specific spectral shifts, and to observe their general or individual specific effect on the virus and infection process; comparing data from in silico, predicted, observed ate molecular step, cellular, animal, and human infection as measured by an evaluative device, clinical, antibody, lymphocyte and immune function data to asymptote toward a progressively more accurate assessment as provided by the novel apparatus of the embodiment of the invention described and disclosed herein, wherein is detected preclinical viral infection as well as a general or individual substance useful in slowing or inhibiting further progression; wherein signatures of the substances described herein are utilized in a known wellness sensor such as a watch or other body sensor or patch clamp device on an in vitro system; wherein an embodiment of the apparatus disclosed herein is constructed as a wearable sensor connected to a smartphone that indicates preclinical viral infection by measuring reaction to and emission of spectral wave patterns and their progressive increases, to warn of infection, stage, risk, and risk of sequellae and constructed including a nano surface receptor coated apparatus that identifies for example without limitation a specific virus in exhaled air or saliva or sputum, to be used for detection of same, and constructed to characterize the totality of the immune response and/or cross reactive attack on infected and non-infected cells, tissues and organs, and the mechanism and inhibition of vicious cycle immune attempts to react that are self-destructive to the infected patient.

In some embodiments of the invention, in particular regarding the aspects of the step of multiple patient data points and/or input and/or iterative input collected, organized, interpreted, processed, structured and/or related and the step of relationships determined between multiple patient data points and the step of applying the determined relationships between multiple data points by the step of formulating and adjusting as needed treatment options, there is utilized a decision engine method and system, as disclosed herein, to provide for example without limitation safe, effective, integrative, holistic, whole-system methods and systems for treating the conditions described herein such as at least, for example and without limitation, acne and other inflammatory skin conditions that provides, inter alia, multiple advantages readily recognized and understood by one of ordinary skill in the art based upon this disclosure, and which decision engine method and system provides solutions to the multiple disadvantages and problems of known methods as described above, in one embodiment, the decision engine method and system provides practical and least-invasive and least-risky paths of diagnosis and treatment and addressing the causal factors of at least, for example and without limitation, acne and other inflammatory skin conditions, and provides symptomatic treatment in a limited fashion when required to reduce risk of morbidity and mortality, and provides the patient with comprehension of the relative benefits and realistic responsibilities of the least-invasive and least-risky paths of diagnosis and treatment that are chosen and wherein the patient carries out those responsibilities, and provides collection of data and/or input regarding events and/or exposure preceding condition onset, along with other historic and/or laboratory parameters, and assessing such data and/or input for etiologic pointers that is directional information as described herein, for example without limitation, including that there are an increase of yeast infections following antibiotics, that there is a worsening of illness and/or onset of other illness following diet, that there is or has been any systemic steroid and/or birth control pill administration including queries for the total months or years of such treatments and which are known to enhance yeast infection.

In some embodiments of the invention, the decision engine method and system provides data and/or input as well as other perspective-driven and sequential patient data points and/or relationships that is entered into an N-dimensional matrix and correlated with, for example without limitation, modification and/or treatment data and/or input and with disease or wellness outcome, and wherein in some embodiments of the invention the N-dimensional matrix is constructed from iterations of one or more steps of the invention such as the steps of collecting multiple patient data points, collecting input from sources described and disclosed herein, organizing such data points and/or input, interpreting such data points and/or input, determining relationships by and between such data points and/or input, applying relationships determined for example by formulating treatment options based on relationships determined, structuring treatment protocols based on treatment options, administering treatment protocol to a patient, monitoring patient response to treatment protocol, wherein for example multiple queries of a patient are interlocking questions utilized to populate the N-dimensional matrix that in part is utilized to assess and evaluate underlying causal factors of acne and/or inflammatory skin conditions and/or inflammation, and/or other disease states of the patient, for example in a particular case where the patient is queried about antibiotic usage following urinary tract infections or surgery and any subsequent bloating or gut issues and any subsequent vaginal yeast infections and any worsening of acne or skin conditions. In some embodiments of the invention, the decision engine method and system provides that assessment and treatment outcomes yielding successful outcomes form a set that is subjected to probability tools such as Bayesian, Monte Carlo, Neural Networks and is compared with other sets for commonality and verification of assumptions and the value of the individual relationship assessments and general healing system perspectives what were imposed. In some embodiments of the invention, the decision engine method and system provides validated tools that are applied to others, wherein is explored exceptions for additional etiologic explanations and contrasted with a lack of validity of the tools. In some embodiments of the invention, the decision engine method and system provides the output following being subjected to probability tools is presented to the physician as well as the patient, to understand it as a team, and such output is then utilized to augment any decision processes, to increase productivity of additional queries, and to provide for utilizing insight and correlation of assembly of a combination of subtle observations, memories, feelings, intuitions.

In yet another embodiment of the invention, in particular regarding the aspects of the step of multiple patient data points collected, organized, interpreted, processed, structured and/or related and the step of relationships determined between multiple patient data points, namely, recognizing and determining relationships between the data points regarding the patient in that a multi-faceted organism such as Candida may have multiple and seemingly opposite effects, in that, for example, Candida overgrowth in the intestines both tolerizes the gut immune system to the large population of Candida, and also causes inflammation, wherein the inflammation due to sensitization to multiple molecular products of the organism causes sensitization to molecular mimics in the skin, such as the Malassezia yeast in the follicles. Thus, the methods and systems of the invention provide safe, effective, integrative, holistic, whole-system methods and systems for treating acne and other inflammatory skin conditions by providing solutions to the multiple disadvantages and problems of known methods as described above, and in one embodiment, by recognizing and determining for example but not limited to the relationships disclosed herein as they relate to multiple patient data points collected, organized, interpreted, processed, structured and/or related.

In yet another embodiment of the invention, in particular regarding the aspects of the step of multiple patient data points collected, organized, interpreted, processed, structured and/or related and the step of relationships determined between multiple patient data points, namely, recognizing and determining relationships between the data points regarding the patient, in cases where there is accompanying autoimmune conditions in the patient such as associated with Epstein-Barr virus and/or Cytomegalovirus, or other unnamed viral episode, by recognizing and determining that administering one or more antivirals, as known by a person of ordinary skill in the art, such as lauricidin that is monolaurin along with administering an algal concentrate such as for example from red algae and/or an algal concentrate containing one or more of an allophycocyanin-rich extract of microalgae such as spirulina and/or chlorella and/or a C-phycocyanin-containing mixture of microalgae such as spirulina and/or chlorella and/or a growth factor-containing mixture derived from microalgae such as spirulina and/or chlorella, is utilized in treatment of dysregulation of immune function due to endogenous release of such viruses, and by recognizing and determining that such administration protects and/or prevents other viruses of particular types that affect the gut and/or gut lining and/or respiratory system, and by recognizing and determining that such administration is helpful in patients whose acne and other inflammatory skin conditions developed after exposure to for example but not limited to enteric viruses such as Coxackievirus and/or flu viruses, and by recognizing and determining that such administration is helpful preventing patient flu infection or flu symptoms at times when vaccines are not available for the particular strains of flu virus circulating, and especially when vaccines do not cover all circulating strains.

In yet another embodiment of the invention, in particular regarding the aspects of the step of multiple patient data points collected, organized, interpreted, processed, structured and/or related and the step of relationships determined between multiple patient data points, namely, recognizing and determining that relationship regarding topical anti-acne products as an optional addition to improve the overall skin appearance following a clear positive experience resulting from treatment by the methods and systems of the invention addressing, inter alia, the underlying causal factors of acne and other inflammatory skin conditions, and that such anti-acne products have improved efficacy because of, for example but not limited to, patient treatment by the inventive methods and systems disclosed herein, and because of the reduction of disease pressure that makes control of acne and other inflammatory skin conditions easier.

In yet another embodiment of the invention, in particular regarding the aspects of the step of multiple patient data points collected, organized, interpreted, processed, structured and/or related and the step of relationships determined between multiple patient data points, namely, recognizing and determining that relationship regarding the interaction, to a degree, of all glands in the patient body, and by extension, all of the systems of the patient body are interactive with regard to conditions toward, or away from, acne and/or other inflammatory skin conditions and/or inflammation in general, and recognizing and determining that relationship regarding, in an embodiment of the invention, one or more of digestion, liver and other eliminative organ functions, microbiome metabolism, hormonal metabolism, are brought into balance when utilizing the inventive method and systems of treatment of the invention at least for the reason they address underlying causal factors of acne and other inflammatory skin conditions, and recognizing and determining that relationship regarding, in an embodiment of the invention, where is sufficient improvements of the underlying causal factors are made so as to see improvement of the acne, then there is improved likelihood of revealing previously-unidentified causal factors that precede acne aggravation and/or other inflammatory skin conditions, and that elimination and/or reduction of these newly-revealed causal factors leads to progressive improvement of the acne and/or other inflammatory skin conditions.

In yet another embodiment of the invention, in particular regarding the aspects of the step of multiple patient data points collected, organized, interpreted, processed, structured and/or related and the step of relationships determined between multiple patient data points, namely, recognizing and determining that relationship regarding anti-microbial herbs that do not destroy normal gut flora, but do target against Candida, for example but not limited to, oregano oil and/or thyme oil and/or sage extract and/or berberine that have beneficial effects including synergistic effects on blood sugar, and/or coptis and/or similar yellow bitter substances, for at least the reason that they not only reduce Candida and proinflammatory bacteria in the intestine, but also inhibit small intestinal bacterial overgrowth, and also are directed against bacteria in the follicle apparatus and/or PSU, while also moderating glucose levels in the blood.

In yet another embodiment of the invention, in particular regarding the aspects of the step of multiple patient data points collected, organized, interpreted, processed, structured and/or related and the step of relationships determined between multiple patient data points, namely, recognizing and determining that relationship regarding utilizing oleaonic acid from Privet fruit to reduce the persistence and/or size of acne cysts and lesions, at least for the reasons of reduction of the effect of leukocyte elastase on attracting more neutrophils into the inflammatory area in the acne lesion.

In other embodiments, as described in the illustrative examples below, an algorithm is constructed from multiple components of the inventive methods and systems including iterative steps herein described intended to collect, organize, interpret multiple data points regarding the patient and to reveal and to determine relevant relationships between multiple data points that are then applied to formulate treatment options that are adjusted as needed and to structure a treatment protocol that is administered to patient to address the underlying causal factors of acne and other inflammatory skin conditions and to monitor patient for response to administered novel treatment protocol and to repeat one or more of these steps, and wherein in some embodiments of the invention the algorithm is constructed from multiple components of the inventive methods and systems includes the so-called human mind computer that is a human medical practitioner utilizing the inventive methods and systems described and disclosed herein and having understanding and/or knowledge as described and disclosed herein so as to access and/or assess and/or evaluate, for example, the complexity of emotional and/or spiritual and/or energetic variables involved in diagnosis and treatment choices.

### EXAMPLES

Various features, aspects, and advantages of the present invention are described below by reference to the following non-limiting examples, which are intended to be illustrative examples. In the illustrative examples below, any and all data based on health information that was derived from patients who were treated has been de-identified and anonymized so that data based on health information has been stripped of all direct identifiers of a patient such it cannot be used to identify a patient. The illustrative examples are representative and are illustrative of specific embodiments of the invention, and are not intended as limiting the scope of the disclosure, as would be recognized and understood by one of ordinary skill in the art. The specification, including the drawings, accompanying claims, and these illustrative examples, provide disclosure of the invention, sufficient to be recognized and understood by one of ordinary skill in the art so that they could make and use of the methods and systems for treating acne and other inflammatory skin conditions, as described and disclosed herein. Many embodiments of the invention can be made and used without departing from the spirit and scope of this disclosure, as recognized and understood by one of ordinary skill in the art. The following illustrative examples describe general conclusions regarding improvement in patient skin conditions, and in particular, as it concerns the variously described aspects and advantages regarding safety and effectiveness of the methods and systems of the invention.

In each case of the illustrative examples described below, as recognized and understood by one of ordinary skill in the art, the invention is shown as providing methods and systems for treating acne and other inflammatory skin conditions, by providing safe, effective, integrative, holistic, whole-system methods and systems of the invention that include, inter alia, the steps of collecting and/or organizing and/or interpreting multiple patient data points, for example but not limited to, patient queries and/or patient histories and/or medical testing and/or observation of patient and/or examination of patient and/or evaluation of patient and/or conventional medicine and/or traditional medicine and/or complementary medicine and/or alternative medicine and/or causal factors of acne and/or causal factors of inflammatory skin conditions, determining relationships between multiple patient data points, applying relationships between multiple patient data, formulating treatment options, adjusting treatment options, structuring a treatment protocol, administering a treatment protocol, monitoring patient for response to administered treatment protocol, optionally reiterating one or more of these steps of collecting, organizing, interpreting, determining, applying, formulating, adjusting, structuring, administering, wherein such acne and other inflammatory skin conditions, when treated using the novel methods and systems of the invention, are improved at least for the reason that the underlying causal factors of the inflammation are also addressed.

### Example One

In Example 1, the initial physical examination of the patient included an assessment of the symptoms and physical signs of the patient, including acne in the creases, and below, the mouth that were diagnosed as peri-oral dermatitis, as well as an interpretation of these symptoms and physical signs as a likely allergic reaction to essential oils such as peppermint that is found in toothpaste. The allergic reaction was interpreted as a result of defective aldehyde oxidase, that is, an enzyme requiring the mineral molybdenum to function. This interpretation was related to the possible nonfunctioning of other molybdenum-dependent enzymes, such as sulfite oxidase, that would result in the patient's difficulty to properly cleave aldehydes and/or sulfites. Another interpretation is that the patient's inability to rid the excess allergens contributed to the sensitivity to those allergens. Follow-up examinations were conducted, utilizing multiple lines of inquiry, including those described below, to explore whether aldehydes and/or sulfites were present in the patient's diet or environment. Multiple data points were collected regarding the patient and relationships were determined between these multiple data points and these relationships were applied in a novel way to develop integrative, useful, safe and efficacious treatment information regarding the patient.

One line of inquiry regarding the patient's history revealed recent purchases of a new car, a new home and new carpeting. This information was interrelated with the observation that these environments are sources, via off-gassing, for volatile organic compounds, including aldehydes such as formaldehyde. A further line of inquiry revealed that the patient used flavored toothpaste containing essential oils such as peppermint. An evaluation of the patient's sensitivity essential oils was conducted using muscle response testing, also called Applied Kinesiology, that indicated weakening to peppermint, cinnamon, lavender, and eucalyptus oils. The patient's history of aggravation of symptoms after exposure and/or use of essential oils reinforced other observations made herein. Although deemed unnecessary in this case because of the likelihood of aggravation of symptoms, it was noted that an additional evaluation, using patch testing for essential oils as contact, could be done for further confirmation of observations made herein. This information was interrelated with knowledge that these essential oils usually contain aldehydes or ketones that are cross-reactive, despite their chemical differences. A further line of inquiry revealed that the patient regularly consumed red wine and hot dogs. This information was interrelated with knowledge that these foods are high in sulfites. The information gathered and observations made were organized to create relationships, including: (i) the high-sulfite foods consumed by the patient depleted any molybdenum present for the proper functioning of sulfite oxidase, (ii) the patient's environment presented high amounts of aldehyde that depleted any molybdenum present for the proper functioning of aldehyde oxidase; (iii) the decreased activity of these enzymes decreased the cleaving of the aldehydes and/or sulfites present, (iv) the increase allergic reaction was a result of un-cleaved aldehydes and/or sulfites. A further line of inquiry revealed that the patient's symptoms worsened after using products containing aldehydes, such as formaldehyde, including: (a) hair relaxer products wherein the formaldehyde might be listed as methylene glycol, formalin, methylene oxide, formic aldehyde, or methanol; (b) paper products such as paper toweling and toilet paper; and (c) cotton fabric wherein the formaldehyde is used as a cross-linking agent; and (d) stay-pressed clothing.

Based on multiple lines of inquiry, several relationships were determined, for example, one relationship determined was between the patient's difficulty to cleave aldehydes and/or sulfites and the patient's acne-like peri-oral dermatitis. That is, the relationship involved the patient's overexposure to certain foods and/or products, as described herein, and the decrease in proper elimination of aldehydes and/or sulfites as a result of molybdenum depletion, and the increase in aldehydes and/or sulfites, that is, the increase in allergens, and the subsequent allergic reaction to those allergens, which was manifested as acneiform contact dermatitis around the mouth where essential oils, like the peppermint in the patient's toothpaste, would contact as often as twice a day. Another relationship determined was between the patient's repeated contact around the mouth with essential oils and the allergens therein (as described herein), and the sensitization of lymphocytes localized in that area resulting from this repeated contact, and the inflammation flare-ups of the acne, and contact and/or exposure to essential oils and/or aldehydes and/or sulfites in the patient's environment such as the air, and in foods ingested by the patient and/or in topical compositions used by the patient. That is, the relationship involved the patient's exposure to allergens such as aldehydes including formaldehyde found in the patient's perfumes and cosmetics, and degassing of such allergens from the carpets and pressboard and glues in the patient's living environment, and releasing from the creams and essential oil diffusers and paper and stay-pressed cotton items that were in contact with the patient. That is, the relationship involved the patient's repeated contact and/or exposure to allergens, and the sensitized lymphocytes localized in the contact area, and the subsequent memory reactions by the patient's immune system.

These relationships were then utilized to create integrative, useful, safe and efficacious treatment information that was the basis for developing a treatment protocol, which addressed the underlying issues, for example, those relating to aldehydes and/or sulfites. The treatment protocol included the patient discontinuing the use of products and the consumption of foods containing aldehydes and/or sulfites, in order to decrease exposure to the allergens found therein, and the patient starting intake of low-dose molybdenum salts, in order to increase the activity of aldehyde oxidase and sulfite oxidase, in order to prevent the allergic reactions due to aldehyde-containing products and/or foods and essential oils by promoting proper degradation and elimination of aldehydes and sulfites. After implementing for a period of time the integrative treatment protocol described herein, including supplementation and greatly-reduced exposure, the patient's condition improved, including reduction of the acne in the creases, and below, the mouth that were diagnosed as peri-oral dermatitis, and eventually all of these symptoms disappeared. Thus, multiple data points were gathered regarding the patient and from this data multiple data relationships were determined and these relationships were then applied in a novel way to develop integrative, useful, safe and efficacious treatment information regarding the patient that was the basis for developing a treatment protocol, which was structured to address the underlying issues and thereby resulted in successfully eliminating the patient's symptoms. Thus, utilization of the methods and systems of the invention successfully treated the patient's condition and provided one or more solutions to the multiple disadvantages and problems of known methods as described above.

### Example Two

In Example 2, multiple data points were collected from the patient and relationships between these data points were determined and then applied by creating treatment information specific to the patient that was then organized to develop an integrative, useful, safe and effective treatment protocol structured to address the underlying issues and thereby resulting in successfully and significantly reduced the patient's cystic acne. This embodiment of the invention describes aspects of the invention wherein the clinical results achieved demonstrate that the novel methods and systems of the invention are effective for treating cystic acne.

A patient with a history of chronic intermittent acne in the past presents with new onset of cystic acne. Multiple patient data points gathered by queries of the patient. They revealed, for example, previous treatment using oral antibiotics, which indicated a repeated and prolonged killing of normal intestinal flora and likely overgrowth of Candida, which are resistant to antibiotics. The initial improvement upon starting antibiotic treatment followed by a worsening afterwards indicated initial suppression of Propionibacterium acnes and/or Cutibacterium acnes followed by a yeast overgrowth leading to inflammation of gut lining. Also, additional patient queries revealed previous treatment using birth control pills and likely immune system modulation such as increased levels of androgen. Patient history of irritable bowel syndrome, and bloating, and abdominal pain after use of antibiotics indicated yeast overgrowth in intestines and likely digestive deficiency (treated with digestive enzyme supplementation) and further examination of tongue implied an intestinal infection (treated with bitter antimicrobial herbs). Also, additional patient queries revealed onset followed celebrations with drinking wine and beer and eating cheeses and sweetened foods such as ice cream, all of which favors tolerance to Candida. Additional patient data included that the majority of past diet for patient was over-processed “fast” foods and foods cooked in high-temperature heated oils. Additional patient data included that there was exposure to chemical allergens from new construction off-gassing and/or old dust and molds. Additional patient data included that there was tenderness in right lower quarter of abdomen and especially over ileocecal valve and positive methane-hydrogen breath test, which indicated the valve likely leaking colon material and bacteria into small intestines (treated by teaching patient massage to close valve, avoid foods that irritate, use antimicrobial herbs, and if necessary, use brief antibiotic regimen). Also, additional patient data included that there was long-term consumption of yeast-free bread, indicating that phytates in unleavened bread may have bound our and depleted essential minerals, followed by testing patient levels and subsequent supplementation with soluble zinc salts.

Thus, after multiple data points were gathered regarding the patient and from this data multiple data relationships were determined, these relationships were then applied in a novel way to develop integrative, useful, safe and efficacious treatment information regarding the patient that was the basis for developing a treatment protocol, which was structured specifically for the patient to address the underlying issues and thereby resulted in successfully eliminating the patient's symptoms. Since a number of suspected causal factors were revealed, they were analyzed for how they interrelate and how much each contributes. For example, the yeast over-growth factor was addressed first to allow the digestive tract immune system time without additional yeast exposure in order to break tolerance to yeast over-growth. Only afterwards, was supplementation with glutamine utilized, to avoid any potential of the glutamine to increase yeast growth in the intestines. Thus, utilization of the methods and systems of the invention successfully treated the patient's condition and provided one or more solutions to the multiple disadvantages and problems of known methods as described above.

### Example Three

In Example 3, an algorithm is constructed from multiple components of the inventive methods and systems including multiple patient queries regarding the causal factors for acne and other inflammatory skin conditions herein described and wherein the patient thereby collected presents multiple decision points depending on the patient's answers to the queries and wherein, at each decision point, additional pathways such as further queries testing is provided, thus creating a branched structure similar to a decision tree such that the collected patient data reveals relevant relationships then applied by creating treatment information specific to the patient that is then organized to develop a treatment protocol structured to address the underlying casual factors related to acne and other inflammatory skin conditions. This embodiment of the invention describes aspects of the invention wherein the methods and systems of the invention may be adapted for use with an algorithm and/or machine learning and/or artificial intelligence that may be used to enhance the collection of patient data points and/or provide options at decision points in the process.

### Example Four

In Example 4, multiple data points were collected from the patient and relationships between these data points were determined and then applied by creating treatment information specific to the patient that was then organized to develop a treatment protocol structured to address the underlying issues and thereby resulting in successfully and significantly reducing the patient's Pityriasis Lichenoides Chronica. This embodiment of the invention describes aspects of the invention wherein the clinical results achieved demonstrate that the novel methods and systems of the invention are effective for treating Pityriasis Lichenoides Chronica.

There was potential for the grade school patient's Pityriasis Lichenoides Chronica condition to progress to lymphoma. Initial queries revealed a history of significant Epstein Barr Virus infection that indicated treatment by antiviral monolaurin, with subsequent significant improvement. Additional queries revealed substantial worsening of symptoms around holidays and the associated large quantities of sugar consumption that indicated a low-sugar, low-yeast diet along with caprylic acid, probiotics, fiber, quercetin, and digestive enzymes. Afterwards, another significant (ninety percent) improvement, and additional favorable result in that there was no need for the previously-planned two-year treatment with methotrexate. Thus, utilization of the methods and systems of the invention successfully treated the patient's condition and provided one or more solutions to the multiple disadvantages and problems of known methods as described above.

### Example Five

In Example 5, multiple data points were collected from the patient and relationships between these data points were determined and then applied by creating treatment information specific to the patient that was then organized to develop a treatment protocol structured to address the underlying issues and thereby resulting in successfully and significantly reducing the patient's symptoms. This embodiment of the invention describes aspects of the invention wherein the clinical results achieved demonstrate that the broadly effective and novel methods and systems of the invention.

A patient with acne, fatigue and Candida, experienced improvement of multiple symptoms at least because of and after adherence to the anti-yeast treatments of the inventive methods and systems of treatment disclosed herein. Additional queries of patient revealed severe cervical dysplasia and biopsies indicating high-risk human papillomavirus (“HPV”), with repeated PAP smears performed. Cervical biopsies at ages 22, 27, and 33 showed a high degree of cervical dysplasia but no cancer, and once on the Candida treatment protocol as described and disclosed herein, administration of a highly purified form of Curcumin and Berberine for six weeks, resulted in normalization of cervix and no high-risk HPV detected and this continuing for more than one year. Afterwards, PAP smear was negative for HPV and APTIMA assay negative. Similarly, a patient with Lichen sclerosis was questioned regarding a partial radical vulvectomy as the result of squamous cell cancer, and also abnormal PAP smears showing virus not usually associated with cancer, but no abnormal HPV. Additional queries revealed that the patient had vitamin D-3 deficiency, mild celiac disease, and Candida. Afterwards, patient underwent a hysterectomy and, upon examination, showed extensive erythema, widespread erosions of different depths, and white areas of atrophy in the vulvar area. Further health history questioning, physical, and Applied Kinesiology testing suggested intestinal Candida over-growth, poor digestion, diminished liver function and pancreatic function and adrenal function, with a likely leaking ileo-cecal valve. Treated as described herein with supplements for these issues, namely, to control intestinal permeability and antigenic leakage leading to general and specific inflammation, as well as a low Candida diet and Berberine and curcumin for viral or possible HPV causing the lichen sclerosis and squamous cell cancer. Treatment protocol resulted in substantial improvements in that there was reduced itching in the vulvar area, much less bloating, and healing of previously-existent plantar fasciitis to the extent that patient could walk two miles on a treadmill at a time. Thus, utilization of the methods and systems of treatment of the invention successfully treated the patient's condition and provided one or more solutions to the multiple disadvantages and problems of known methods as described above.

### Example Six

In Example 6, multiple data points were collected from the patient and relationships between these data points were determined and then applied by creating treatment information specific to the patient that was then organized to develop an integrative, useful, safe and effective treatment protocol structured to address the underlying issues and thereby resulting in successfully and significantly reduced the patient's cystic acne. This embodiment of the invention describes aspects of the invention wherein the clinical results achieved demonstrate that the novel methods and systems of the invention are effective for treating the patient condition described herein.

This embodiment of the invention describes aspects of the invention wherein the clinical results achieved demonstrate that the novel methods and systems of the invention are effective for treating acne. Specifically, a patient with a history of acne and multiple patient data points gathered at least by multiple queries of the patient which reveal, for example, patient history of causal factors, namely aggravators such as yeast, gluten, and specific foods such as high sugar foods, stress, leaky gut, hormonal issues, toxicity, genetic single nucleotide polymorphisms affecting metabolic breakdown and/or elimination, external sensitizers and/or irritants such as topical cutting oils leading to chloracne, repeated use of antibiotics when young for otitis and later in childhood for urinary tract infections, gut disturbances, yeast infections, flare-up of acne following cessation of antibiotics, history of using non-steroidal anti-inflammatory drugs, history of using protein pump inhibitors, which preceded aggravation of gut symptoms and/or acne, and which disturbed secretion of the mucoid coat lining the intestinal wall and leading to leaky gut. Relationships were determined between these multiple data points and determined relationships were applied by formulating and adjusting treatment options based on at least multiple data points including, history highly suggestive of Candida overgrowth, and weakness of tensor fascia lata or muscle response testing when touching over L1, L2 based on Chinese medicine, as readily recognized and understood by one of ordinary skilled in the art, and strengthened by anti-yeast supplement or medications placed in energy field such as under the south pole of a strong flat magnet over the solar plexus, and formulating a high vegetable and low glycemic diet for structuring a treatment protocol to be administered, and formulating elimination of the known and suspected allergens and structuring a treatment protocol to be administered, and administering a treatment protocol that also included trial avoidance of suspected foods related to acne inflammation such as fried food, junk food, dairy products, other pro-inflammatory foods, excessive toxins, high animal fat, excessive stimulants such as for example without limitation, coffee, high glycemic foods and sugar.

Reiterating the step of collecting, organizing, interpreting and relating multiple data points by further patient queries regarding daily bowel movements and revealing less-than-optimal schedule for same, and adjusting treatment options to include high fiber diet to increase frequency, as well as administering one or more of the following as needed, including high vitamin C in water regimens Epsom salts, enemas, colonics, or combinations of Ayurvedic traditions herbs such as triphala with other substances such as magnesium citrate, Emblica officinalis, Terminalia chembula, Terminalia belerica, and/or Aloe ferox. Determined relationships between multiple data points were applied by formulating and adjusting treatment options including improving digestion and improve ability to tolerate yeast die-off symptoms, including utilizing soluble fiber in diet, quercetin, digestive enzymes, liver support supplements, specific detoxification as needed as physical and/or testing indicates, caprylic acid, probiotics, as well as avoiding exposure to yeast, mold, foods and supplements made by or with these organisms and containing their breakdown products such as mushrooms, moldy rooms or buildings or locations with centralized air conditioning, and determining the relationship regarding all of these leading to tolerization of the immune system to yeast and molds. Applying determined relationships between multiple data points by formulating and adjusting as needed treatment options that formed the basis for structuring a treatment protocol administered to patient, including, after monitoring for reduction of yeast population, addressing and/or repairing leaky gut wherein the gut immune system is reinstructed to control Candida population at a lower level despite possible glycemic effect of glutamine supplementation, and adjust if needed by testing stool for Candida presence, species, and sensitivity, and for inflammatory bacteria and parasites, and testing discharge or white material on tongue under microscope for pseudo-hyphae, and formulating treatment options and structuring treatment protocols such as treating with other anti-yeast herbs, or if necessary, with nystatin, and using glutamine and mucoid substances swallowed to coat and to restore the integrity of the cells lining the small intestines to repair leaky gut.

Monitoring patient after administrations of treatment protocols by reiterating step of collecting, organizing, interpreting multiple patient data points relating to feedback from patient revealed patient repeated touching, picking, squeezing, instrumentation, or manipulation of lesions, which indicated formulating treatment options including substitute habits, reminders, powerful emotional rehabilitation, emotionally-focused therapy, behavior modification techniques to assist with stopping such behavior. Additional formulations and adjustments to treatment options forming the basis for treatment protocol includes using supplements to correct hormonal imbalances, calcium-D-glucarate to lower estrogens, pseudo-estrogens and other toxins that are carried out by glucuronidation, using Vitex castus agnes that is chaste tree to balance follicle stimulating hormone and luteinizing hormone and thus estrogen and progesterone, and to balance prolactin, using pyridoxal-5-phosphate for premenstrual aggravation, increased in at time of aggravation in menstrual cycle, and/or indole-3-carbinol to re-balance estrogen subtypes, as well as using additional herbal products that remove aggravating substances from the skin by supporting lymphatic drainage, eliminative organ function, or clearing at the subcellular organelle level, such as lysosomal emptying, such as burdock root and seed and other herbal products, some of which are rich in saponins, soap-like molecules able to disrupt membrane structure of sub-cellular collecting organelles such as lysosomes, and possibly of lipopolysaccharides the cell membranes of gram-negative bacteria from, for example, the colon, and which have been associated with other inflammatory skin diseases such as psoriasis. Lastly, reiterating steps of formulating treatment options as basis for structuring optional treatment protocols administered after above steps were completed and the underlying causal factors were addressed, which options included for example topical treatments such as acne surgery, gritty soap, or retinoids to remove plugs in follicles, bentonite clay mixed with gotu kola, supplements from regimen used internally in this regimen mixed with aloe vera, witch hazel, or water depending on skin sensitivity and oiliness, placed on herbs overnight, or a tea-tree oil mix in skin penetrating oils applied to lesions as an anti-microbial. Thus, utilization of the methods and systems of the invention successfully treated the patient's condition and provided one or more solutions to the multiple disadvantages and problems of known methods as described above.

### Example Seven

In Example 7, multiple data points were collected from the patient and relationships between these data points were determined and then applied by creating treatment information specific to the patient that was then organized to develop an integrative, useful, safe and effective treatment protocol structured to address the underlying issues and thereby resulting in successfully and significantly reduced the patient's cystic acne. This embodiment of the invention describes aspects of the invention wherein the clinical results achieved demonstrate that the novel methods and systems of the invention are effective for treating the patient condition described herein.

A patient with a history of acne complicated by digestive issues, chronic fatigue and multiple autoimmune disorders is queried to collect and organize multiple patient data points that are also interpreted and related. The multiple data points were collected from the patient and relationships between these data points were determined and then applied by creating treatment information specific to the patient that was then organized to develop an integrative, useful, safe and effective treatment protocol structured to address the underlying causal factors of the acne and other conditions presented by the patient and thereby resulting in successfully and significantly reducing the patient's acne and other conditions as described herein. This illustrative example embodiment of the invention describes aspects of the invention wherein the clinical results achieved demonstrate that the novel methods and systems of the invention are effective.

Multiple data points were collected from the patient, included that patient had been treated with multiple supplements and IV vitamin C and minerals, that physical examination revealed over sixty fine papules and pustules on the face, and scattered papules and pustules on her back, that patient ate primarily a junk food diet, that patient had taken antibiotics and had two previous yeast infections. Relationships were determined between the multiple data points and applied to formulating treatment options that included a yeast free diet, soluble fiber supplements, quercetin, digestive enzymes, liver supportive herbs, capryllic acid to kill yeast, probiotics, and a glutamine product to repair leaky gut, which options were used to structure the treatment protocol that was administered to patient, whereupon there was notable improvement of patient acne. However, in the years monitored, the patient was treated by other physicians with antibiotics for infections and/or procedures, and at times did not follow dietary treatment protocols, which resulted in bloating, low energy, and acne breakouts, which symptoms, however improved each time with resumed administration of the treatment protocol based on the methods and systems of the invention as disclosed herein. The step of collecting multiple data points was reiterated whereupon was revealed a history of mononucleosis when a preteen and which revealed upon review of lab data, markedly increased anti-nuclear antibody levels and other dramatically elevated tests for autoimmunity, and very elevated Epstein bar virus and cytomegalovirus titers. Determined relationships of the multiple data points were applied by formulating treatment options including reducing yeast overgrowth in the gut as one basis for structuring a treatment protocol that was administered to patient with resulting improvement in acne and overall condition.

Reiteration of the step of collecting, organizing, interpreting and relating multiple data points was done, including review and analysis of lab data showing strong parameters of autoimmunity and evidence of two viruses that have been associated with inducing autoimmunity, which was followed by reiterating the step of determining relationships between multiple data points and applying determined relationships by formulating and adjusting treatment options to structure a treatment protocol that included administration of monolaurin, a coconut oil derivative with strong anti-viral activity that kills released virus by interfering with the lipid coat, and which greatly reduced the circulating Epstein bar virus in the bloodstream, and which treatment protocol included administration of an allophycocyanin-rich highly concentrated extract of spirulina based upon recognition and determination of the relationship between this extract and prevention of viral entry into the cell leading to more virus in the bloodstream available for attack by the antiviral monolaurin and subsequent synergistic effects. The administered synergistic combination of antiviral and allophycocyanin-rich highly concentrated extract of spirulina reduced and/or eliminated the viral-based dysregulation of the patient's immune response that led to inappropriate responses against self-tissues, including against molecular mimics in the sebaceous glands and perifollicular apparatus, leading to the inflammation seen as acne. Afterwards, the step of monitoring revealed the patient experienced an increase in energy and wellbeing and improvement to the acne, with a reduction in the number, size, and frequency of acne lesions, and after some time of noncompliance with the treatment protocol, the patient acne flared up, but again was improved upon re-administration of the treatment protocol as described herein. Thus, utilization of the methods and systems of the invention successfully treated the patient's condition and provided one or more solutions to the multiple disadvantages and problems of known methods as described above. Notably, the patient progressed from an inability to work regularly to having a dramatic increase in energy and the ability to work at a job requiring ten hour days for six days per week.

### Example Eight

In Example 8, multiple data points are collected from the patient and relationships between these data points are determined and applied by creating treatment information specific to the patient that is organized to develop an integrative, useful, safe and effective treatment protocol structured to address the underlying issues and thereby resulting in successfully and significantly reduced the patient's acne and other inflammatory skin conditions, as well as chronic fatigue. This embodiment of the invention describes aspects of the invention wherein the unprecedented clinical results demonstrate that the novel methods and systems of the invention are safe and effective for treating the patient condition described herein. Specifically, multiple data points are collected from the patient who was a young Afro-American female with chronic intermittent Urticaria for multiple years and who had been treated by a previous physician with at least two different antihistamines without improvement in her condition. Multiple queries of the patient were conducted to collect multiple patient data points, including for example patient history suggesting yeast overgrowth in the intestines, use of essential oils on a regular basis, brushing teeth regularly with mint toothpaste. Relationships are determined between the multiple data points and applied to formulating treatment options and selecting a treatment protocol that included, for example, a yeast-free diet and digestive support as described herein, elimination of the use of essential oils, and with improvement after this treatment protocol was administered, then the treatment protocol was adjusted to include cessation of the antihistamines and addition of administering molybdenum salts about 500 micrograms daily based in part on understanding and knowledge of molybdenum salts as cofactors aldehyde oxidase, an enzyme that breaks down aldehydes, and which are related to ketones, and which are found in many essential oils. Following compliance with the treatment protocol, there was significant improvements of the patient's Urticaria. Thus, utilization of the methods and systems of the invention successfully and effectively and safely treat patient's conditions and provide one or more solutions to the multiple disadvantages and problems of known methods above.

### Example Nine

In Example 9, multiple data points are collected from the patient and relationships between these data points are determined and applied by creating treatment information specific to the patient that is organized to develop an integrative, useful, safe and effective treatment protocol structured to address the underlying issues and thereby resulting in successfully and significantly reduced the patient's acne and other inflammatory skin conditions, as well as chronic fatigue. This embodiment of the invention describes aspects of the invention wherein the unprecedented clinical results demonstrate that the novel methods and systems of the invention are safe and effective for treating the patient condition described herein. Specifically, multiple data points are collected from the patient, including that the patient was previously treated without success and/or improvement. The patient after physical examination revealed acne papules and pustules on the face and scattered papules and pustules on the back and additional queries provided input such as that the patient primarily ate a junk food diet and that the patient had taken antibiotics as part of previous unsuccessful treatments and had experienced two yeast infections. Relationships are determined between the multiple data points and applied to formulating treatment options that included, for example, a yeast-free diet, soluble fiber supplements, quercetin, digestive enzymes, liver supportive herbs, capryllic acid to kill the yeast, probiotics, a glutamine product to repair leaky gut, which are used to structure the treatment protocol that is administered to patient, whereupon there was notable improvements of the patient's acne and other inflammatory skin conditions as well as chronic fatigue. The step of collecting multiple data points is reiterated whereupon was revealed further data and input from the patient and additional determined relationships of the multiple data points were applied by formulating treatment options including, for example, reducing yeast overgrowth in the gut as one basis for structuring a treatment protocol that was administered to patient with resulting improvement in acne and other inflammatory skin conditions and overall condition of patient physically, emotionally, and spiritually.

Reiteration of the step of collecting, organizing, interpreting and relating multiple data points is done, including input from various medicines and/or various healing systems such as conventional medicine, traditional medicine, complementary medicine, alternative medicine, functional medicine, restorative medicine, precision medicine, causal factors of acne and/or acne inflammation and other inflammatory skin conditions, functional medicine, restorative medicine, precision medicine. Additional multiple data points are collected from the patient and relationships between these data points are determined and applied after review and analysis of lab data showing strong parameters of autoimmunity and evidence of two viruses associated with inducing autoimmunity, which is followed by reiterating the step of determining relationships between multiple data points and applying determined relationships by formulating and adjusting treatment options to structure a treatment protocol that included administration of multiple herbs, minerals, antivirals as described herein and the subsequent synergistic effects, which administered synergistic combination, especially from antivirals and allophycocyanin-rich highly concentrated extract of spirulina provides an integrative, useful, safe and effective treatment protocol structured to address the underlying issues. Afterwards, the step of monitoring reveals the patient experienced an increase in energy and wellbeing and improvement to the acne, including reduction in the number, size, and frequency of acne lesions, and after some time of noncompliance with the treatment protocol resulting in the patient experiencing an acne flare up, improvement is again seen upon re-administration of the treatment protocol as described herein. Thus, utilization of the methods and systems of the invention successfully and effectively and safely treat patient's conditions and provide one or more solutions to the multiple disadvantages and problems of known methods as described above.

While the foregoing disclosure, including the specification, and the illustrative examples, and the drawings, represent various embodiments of the invention, it is understood and recognized by one of ordinary skill in the art that various additions, modifications and substitutions may be made by one of ordinary skill in the art without departing from the spirit and scope of the principles of the invention, which is defined in the accompanying claims. These various and specific embodiments are therefore to be considered in all respects as illustrative and as examples and as not restrictive and as not limiting the invention. Many possible embodiments of the invention may be made and used and will be readily evident one of ordinary skill in the art upon examination of the disclosure of the invention herein. As recognized and understood by one of ordinary skill in the art, there are numerous features and sub-combinations of the invention that may be made and used without reference to other features and sub-combinations. Various objects, features, aspects and advantages of the invention will be readily evident to one of ordinary skill in the art upon examination of the description of the invention herein, including the specification, and the illustrative examples, and the drawings, and the appended claims.

What is claimed is:

1. A method for the treatment of acne, the method comprising the steps of:
collecting multiple patient data points;
organizing multiple patient data points;
interpreting multiple patient data points;
determining relationships between the multiple patient data points;
applying relationships between the multiple patient data points by formulating treatment options based on relationships between the multiple patient data points and optionally adjusting treatment options based on relationships between the multiple patient data points;
structuring a treatment protocol based on treatment options;
administering treatment protocol to a patient; and
monitoring patient response to treatment protocol;
wherein the steps of collecting, organizing, interpreting, determining, applying, formulating, adjusting, structuring, are done in view of causal factors of acne and other inflammatory skin conditions; and
wherein one or more steps of collecting, organizing, interpreting, determining, applying, formulating, adjusting, structuring, monitoring are iterated.

2. The method of claim 1 wherein the patient data points include one or more of the following: one or more patient queries, patient history, medical testing, observation of the patient, examination of the patient, evaluation of the patient, conventional medicine, traditional medicine, complementary medicine, alternative medicine, causal factors of acne, and causal factors of inflammatory skin conditions.

3. The method of claim 2 wherein the patient queries include one or more of the following: patient exposure to environmental toxins, patient stress, patient diet, patient digestion, patient antibiotic use, and patient birth control pill use.

4. The method of claim 1 wherein the step of determining relationships between the multiple patient data points includes one or more of the following patient data points: patient intestinal yeast overgrowth, patient poor digestion, patient leaky gut, patient antibiotic usage, patient birth control pill usage, gluten levels in patient diet, sugar levels in patient diet, and fiber levels in patient diet.

5. The method of claim 1 wherein the step of applying relationships between the multiple patient data points by formulating treatment options includes one or more of the following treatment options: reducing patient intestinal yeast overgrowth, improving patient digestion, repairing patient leaky gut, reducing patient antibiotic usage, reducing patient birth control pill usage, reducing gluten levels in patient diet, reducing sugar levels in patient diet, increasing fiber levels in patient diet, decreasing ileo-cecal valve backflow, increasing patient liver function.

6. The method of claim 1 wherein the treatment options include on or more of the following: reducing patient intestinal overgrowth using one or more of the following: reducing sugar in patient diet, reducing patient junk food diet, and increasing prebiotics in patient diet.

7. The method of claim 1 wherein the treatment options include repairing patient leaky gut using one or more of the following: reducing gluten in patient diet, reducing incompletely digested foods in patient, reducing patient use of non-steroidal anti-inflammatory drugs, reducing patient use of proton pump inhibitors.

8. The method of claim 1 wherein the treatment options include enhancing patient aldehyde oxidase activity using one or more of the following: low-dose molybdenum salts, reducing patient exposure to aldehydes in foods, and reducing patient exposure to aldehydes in patient's environment.

9. The method of claim 1 wherein the treatment options include an antiviral combined with at least one or more of the following: an allophycocyanin-rich extract of microalgae, C-phycocyanin-containing mixture of microalgae, and a growth factor-containing mixture derived from microalgae such as spirulina and/or chlorella.

10. The method of claim 9 wherein the antiviral is lauricidin and the microalgae is selected from the group consisting of: spirulina and chlorella.

11. A method for the treatment of inflammatory skin conditions, the method comprising the steps of:
collecting multiple patient data points;
organizing multiple patient data points;
interpreting multiple patient data points;
determining relationships between the multiple patient data points;
applying relationships between the multiple patient data points by formulating treatment options based on relationships between the multiple patient data points and optionally adjusting treatment options based on relationships between the multiple patient data points;
structuring a treatment protocol based on treatment options;
administering treatment protocol to a patient; and
monitoring patient response to treatment protocol;
wherein the steps of collecting, organizing, interpreting, determining, applying, formulating, adjusting, structuring, are done in view of causal factors of acne and other inflammatory skin conditions; and
wherein one or more steps of collecting, organizing, interpreting, determining, applying, formulating, adjusting, structuring, monitoring are iterated.

12. The method of claim 11 wherein the patient data points include one or more of the following: one or more patient queries, patient history, medical testing, observation of the patient, examination of the patient, evaluation of the patient, conventional medicine, traditional medicine, complementary medicine, alternative medicine, causal factors of acne, and causal factors of inflammatory skin conditions.

13. The method of claim 11 wherein the patient queries include one or more of the following: patient exposure to environmental toxins, patient stress, patient diet, patient digestion, patient antibiotic use, and patient birth control pill use.

14. The method of claim 11 wherein the step of determining relationships between the multiple patient data points includes one or more of the following patient data points: patient intestinal yeast overgrowth, patient poor digestion, patient leaky gut, patient antibiotic usage, patient birth control pill usage, gluten levels in patient diet, sugar levels in patient diet, and fiber levels in patient diet.

15. The method of claim 11 wherein the step of applying relationships between the multiple patient data points by formulating treatment options includes one or more of the following treatment options: reducing patient intestinal yeast overgrowth, improving patient digestion, repairing patient leaky gut, reducing patient antibiotic usage, reducing patient birth control pill usage, reducing gluten levels in patient diet, reducing sugar levels in patient diet, increasing fiber levels in patient diet, decreasing ileo-cecal valve backflow, increasing patient liver function.

16. The method of claim 11 wherein the treatment options include on or more of the following: reducing patient intestinal overgrowth using one or more of the following: reducing sugar in patient diet, reducing patient junk food diet, and increasing prebiotics in patient diet.

17. The method of claim 11 wherein the treatment options include repairing patient leaky gut using one or more of the following: reducing gluten in patient diet, reducing incompletely digested foods in patient, reducing patient use of non-steroidal anti-inflammatory drugs, reducing patient use of proton pump inhibitors.

18. The method of claim 11 wherein the treatment options include enhancing patient aldehyde oxidase activity using one or more of the following: low-dose molybdenum salts, reducing patient exposure to aldehydes in foods, and reducing patient exposure to aldehydes in patient's environment.

19. The method of claim 11 wherein the treatment options include an antiviral combined with at least one or more of the following: an allophycocyanin-rich extract of microalgae, and a growth factor-containing mixture derived from microalgae such as spirulina and/or chlorella.

20. A method for the treatment of inflammation, the method comprising the steps of:
collecting multiple patient data points;
organizing multiple patient data points;
interpreting multiple patient data points;
determining relationships between the multiple patient data points;
applying relationships between the multiple patient data points by formulating treatment options based on relationships between the multiple patient data points and optionally adjusting treatment options based on relationships between the multiple patient data points;
structuring a treatment protocol based on treatment options;
administering treatment protocol to a patient; and
monitoring patient response to treatment protocol;
wherein the steps of collecting, organizing, interpreting, determining, applying, formulating, adjusting, structuring, are done in view of causal factors of acne and other inflammatory skin conditions; and
wherein one or more steps of collecting, organizing, interpreting, determining, applying, formulating, adjusting, structuring, monitoring are iterated.
